(19) United States
(12) Patent Application Publication
(10) Pub. No.: US 2026/0182552 A1
(43) Pub. Date: Jul. 2, 2026

(54) SYSTEM FOR STORING AND RELEASING BENEFICIAL ARTHROPODS AND METHODS FOR ITS USE AND PRODUCTION

(71) Applicant: Koppert B.V., BE Berkel en Rodenrijs (NL)

(72) Inventors: Diede Nina MELSEN, BE Berkel en Rodenrijs (NL); Thomas Volkert Marie GROOT, BE Berkel en Rodenrijs (NL); Radbout TIMMER, BE Berkel en Rodenrijs (NL); Tromp Cornelis Willem BAKKER, BE Berkel en Rodenrijs (NL); Johanna Alida WECHGELAAR, BE Berkel en Rodenrijs (NL); Cornelia Jozina Maria REMIJN, BE Berkel en Rodenrijs (NL); Leonie Christina VAN DEN BERG, BE Berkel en Rodenrijs (NL)

(21) Appl. No.: 19/126,024

(22) PCT Filed: Oct. 31, 2023
(86) PCT No.: PCT/NL2023/050571

§ 371 Date: Apr. 30, 2025

(30) Foreign Application Priority Data
Nov. 1, 2022 (NL) 2033437

(51) Int. Cl. A01K 67/31; A01G 13/10

(52) U.S. Cl. CPC A01K 67/31; A01G 13/10

(57) ABSTRACT

The present invention according to a first aspect relates to a system for releasing beneficial arthropods. Further aspects relate to a method for biological pest control and the use of the system in pest control. The invention furthermore relates to a system for storing beneficial arthropods and a method for producing such a system. All aspects of the invention have utility in biological pest control, in particular in crop protection.

### CROSS REFERENCE TO RELATED APPLICATIONS

This application is a U.S. National Phase filing under 35 U.S.C. § 371 of International Application PCT/NL2023/050571, filed Oct. 31, 2023, and published as WO 2024/096734 A1 on May 10, 2024. PCT/NL2023/050571 claims priority from The Netherlands application number 2033437, filed Nov. 1, 2022. The entire contents of each of these prior applications are hereby incorporated herein by reference.

### BACKGROUND

The present invention relates to the field of biological pest control using beneficial arthropod.

### SUMMARY OF THE INVENTION

In view of the clear downsides of the use of chemicals to control insect and arachnid pests the use of natural enemies of such pests as biological controlling agents has increased over the last decades. Many of these natural enemies are predatory and/or parasitic arthropods. For effectively employing them on a large scale for pest control it is of high importance to efficiently produce, store (in particular during transport) and release (distribute) them in the target area where the pest is present. In the area of the production of many of these predatory and/or parasitic arthropods (beneficial arthropods) many important developments have been made. The present invention focuses on the development of new systems for storing and releasing beneficial arthropods, such as predatory arthropods and parasitic arthropods.

For this the present invention according to a first aspect provides a system for releasing beneficial arthropods comprising an arthropod housing comprising a body, preferably a plate formed body, provided with a number of chambers enclosing an interior space, wherein the shape of the interior space of the number of chambers is such that the dimensions in at least one direction are smaller than the dimensions in at least one of the other two direction, wherein each chamber has a number of openings suitable to provide access of the arthropods to the interior space. The chambers of the arthropod housing comprise individuals, preferably motile individuals, of a population of a beneficial arthropod species.

According to a further aspect, the present invention relates to a method for biological pest control, preferably in crop protection, comprising providing a number of systems for releasing beneficial arthropods according to the invention to a target area.

According to a further aspect, the present invention relates to the use of the system for releasing beneficial arthropods of the invention for pest control.

According to a further aspect, the present invention relates to a system for storing and/or releasing beneficial arthropods, said system comprising:
I. a container having a base and a number of walls extending from the base, said number of walls surrounding a void;
II. a number of arthropod release systems according to claims 1-8 positioned in the void;
III. preferably a food source for the individuals of the beneficial arthropod species;
IV. preferably a water source suitable for the individuals of the beneficial arthropod population, optionally together with a separation means, suitable to separate the water source in a separated part of the void;
V. closing means closing the void, preferably closing means cooperating with a number of the walls.

According to yet a further aspect, the present invention relates to a method for producing a system for storing and/or releasing beneficial arthropods, said method comprising:
A. providing a container having a base and a number of walls extending from the base, said walls surrounding a void;
B. providing a number of arthropod housing devices of the invention;
C. providing individuals, preferably motile individuals, of a population of the beneficial arthropod, optionally on a carrier material;
D. preferably providing a food source for the individuals of the beneficial arthropod species and placing the food source in the void;
E. preferably providing a water source suitable for the individuals of the beneficial arthropod population optionally together with a separation means, suitable to separate the water source in a separated part of the void and placing the water source in the void;
F. placing the number of arthropod housing devices in the void;
G. placing the individuals of the beneficial arthropod population, optionally on the carrier material, in the void;
H. providing closing means suitable to close the void and closing the void with said closing means.

### BRIEF DESCRIPTION OF THE FIGURES

FIGS. 1-9 provide an abstract overview of different process steps of the method for producing the system for storing and/or releasing beneficial arthropods.

FIG. 10 presents an embodiment of the system for releasing beneficial arthropods in its use in a crop plant.

FIGS. 11 and 12 present graphical data from experiment 6.

### DETAILED DESCRIPTION

The system for releasing beneficial arthropods of this invention is suitable for releasing beneficial arthropods in a target area. Within the context of the present invention, the term “beneficial arthropods” should be considered to mean arthropods from a species that can be used advantageously. Whenever in the context of the present invention the term arthropod is used, it is intended to refer to beneficial arthropods, unless specifically stated differently. Beneficial arthropods may for example be biocontrol agents (natural enemies) of insect pests or arachnid pests, such as pests for crops, agricultural livestock or other animals. Examples of beneficial arthropods used as biocontrol agents are predatory insects, parasitic wasps and predatory mites. According to the invention the beneficial arthropods most preferably are biological control agents (BCAs).

Arthropod prey, such as Astigmatid mites, for predatory arthropods, such as predatory mites or predatory insects, may also be beneficial in a target area where the predatory arthropods are present. This provision of arthropod prey to the predatory arthropods may help in supporting the development and maintenance of the population of predatory arthropods (see for example Hoogerbrugge et al. (2008), Integrated Control in Protected Crops, Temperature Climate, IOBC/wprs Bulletin Vol 32, pp. 79-82 and EP16154905).

The present invention has broad utility for storing and releasing beneficial arthropods selected from predatory insects, parasitic wasps, predatory mites and arthropod prey, in different target areas, depending on their application. Beneficial arthropods that may suitably be stored and distributed with the present invention may be selected broadly across various families from different taxonomical orders. Below various embodiments will be disclosed with selections of beneficial arthropods from different families. In these lists presented below preferred groups for sections of beneficial arthropods are presented in underlined font and more preferred selections are presented in bold and underlined font.

Within the order Coleoptera suitable predatory beneficial arthropods may be selected from:
the family Coccinellidae, such as from the genus Adalia, e.g. Adalia bipunctata (Linnaeus); from the genus Chilocorus, e.g. Chilocorus baileyi (Blackburn), Chilocorus bipustulatus (Linnaeus), Chilocorus circumdatus Gyllenhal, Chilocorus nigrita (Fabricius); from the genus Clitostethus, e.g. Clitostethus arcuatus (Rossi); from the genus Coccidophilus, e.g. Coccidophilus citricola Brethes; from the genus Coccinella, e.g. Coccinella septempunctata Linnaeus; from the genus Cryptolaemus, e.g. Cryptolaemus montrouzieri Mulsant; from the genus Cycloneda, e.g. Cycloneda limbifer Casey; from the genus Delphastus, e.g. Delphastus catalinae Horn (syn. Delphastus pusillus LeConte)); from the genus Diomus; from the genus Exochomus, e.g. Exochomus laeviusculus Weise, Exochomus quadripustulatus (Linnaeus); from the genus Harmonia, e.g. Harmonia axyridis Pallas; from the genus Hippodamia, e.g. Hippodamia convergens Guérin-Méneville; from the genus Leis, e.g. Leis (Harmonia) dimidiata (Fabricius); from the genus Lemnia, e.g. Lemnia biplagiata (Schwartz); from the genus Nephus, e.g. Nephus includens (Kirsch), Nephus quadrimaculatus (Herbst), Nephus reunioni Fursch; from the genus Propylea, e.g. Propylea japonica (Thunberg), Propylea quatuordecimpunctata (Linnaeus); from the genus Rhyzobius, e.g. Rhyzobius chrysomeloides (Herbst), Rhyzobius forestieri (Mulsant), Rhyzobius lophanthae (Blaisdell), Rodolia cardinalis (Mulsant); from the genus Scymnus, e.g. Scymnus rubromaculatus (Goeze), Scymnus interruptus; from the genus Stehtorus, e.g. Stethorus punctillum Weise:
the family Staphylinidae, such as from the genus Aleochara, e.g. Aleochara bilineata (Gyllenhaal); from the genus Dalotia, e.g. Dalotia (Atheta) coriaria Kraatz, from the genus Holobus, e.g. Holobus flavicornis (Boisduval & Lacordaire); from the genus Atheta.

Within the order Diptera suitable predatory beneficial arthropods may be selected from:
the family Cecidomyiidae, such as from the genus Aphidoletes, e.g. Aphidoletes aphidimyza (Rondani), from the genus Feltiella, e.g. Feltiella acarisuga (Vallot), from the genus Coenosia, e.g. Coenosia attenuata Stein, from the genus Hydrothea, e.g. Hydrothea aenescens (Wiedemann);
the family Syrphidae, such as from the genus Episyrphus, e.g. Episyrphus balteatus Geer, such as from the genus Sphaerophoria, e.g. Sphaerophoria rueppellii (Wiedemann), from the genus Eupeodes, e.g. Eupeodes corollae.

Within the order Hemiptera suitable predatory beneficial arthropods may be selected from:
the family Anthocoridae, such as from the genus Anthocoris, e.g. Anthocoris nemoralis (Fabricius), Anthocoris nemorum (Linnaeus), from the genus Orius, e.g. Orius albidipennis (Reuter), Orius insidiosus (Say), Orius laevigatus (Fieber), Orius majusculus, Orius sauteri (Poppius), Orius strigicollis (Poppius) or Orius tristicolor (White);
the family Lygaidae, such as from the genus Geocoris, e.g. Geocoris punctipes;
the family Miridae, such from the genus Dicyphus, Discopus errans (Wolff) or Discopus hesperus Knight, from the genus Macrolophus, e.g. Macrolophus pygmaeus (Rambur), from the genus Nesidiocoris, e.g. Nesidiocoris tenuis (Reuter);
the family Nabidae, such as from the genus Nabis, e.g. Nabis pseudoferus ibericus Remane;
the family Pentatomidae, such as from the genus Picromerus, e.g. Picromerus bidens Fabricius or Podisus maculiventris (Say).

From the order Hymenoptera beneficial arthropods may be selected from:
the family Ampulicidae, such as from the genus Ampulex, e.g. Ampulex compressa (Fabricius);
the family Aphelinidae, such as from the genus Aphelinus, e.g. Aphelinus abdominalis (Dalman), Aphelinus asychis (Walker), Aphelinus mali (Haldeman), Aphelinus varipes (Forster), from the genus Aphytis, e.g. Aphytis diaspidis (Howard), Aphytis holoxanthus DeBach, Aphytis lepidosaphes Compere, Aphytis lingnanensis Compere, Aphytis melinus DeBach, from the genus Cales, e.g. Cales noacki Howard, from the genus Coccophagus, e.g. Coccophagus cowperi Girault, Coccophagus gurneyi Compere, Coccophagus lycimnia (Walker), Coccophagus pulvinariae Compere, Coccophagus rusti Compere, Coccophagus scutellaris (Dalman), from the genus Encarsia, e.g. Encarsia citrina (Craw), Encarsia formosa Gahan, Encarsia guadeloupae Viggiani, Encarsia brasiliensis (Hempel) (=hispida De Santis), Encarsia perniciosi (Tower), Encarsia protransvena Viggiani, Encarsia tricolor Forster, from the genus Eretmocerus, e.g. Eretmocerus eremicus Rose & Zolnerowich, Eretmocerus mundus Mercet;
the family Bethylidae, such as from the genus Cephalonomia, e.g. Cephalonomia tarsalis (Ashmead);
the family Braconidae, such as from the genus Aphidius, e.g. Aphidius colemani Viereck, Aphidius ervi (Haliday), Aphidius gifuensis Ashmead, Aphidius matricariae Haliday, Aphidius urticae Haliday, from the genus Bracon, e.g. Bracon brevicornis Wesmael, from the genus Cotesia, e.g. Cotesia flavipes, Cotesia glomerata (Linnaeus), Cotesia rubecula (Marshall), from the genus Dacnusa, e.g. Dacnusa sibirica Telenga, from the genus Ephedrus, e.g. Ephedrus cerasicola Stary, Ephedrus plagiator (Nees), from the genus Habrobracon, e.g. Habrobracon hebetor (Say), from the genus Lysiphlebus, e.g. Lysiphlebus fabarum (Marshall), from the genus Meteorus, e.g. Meteorus bakeri Cook & Davis, Meteorus gyrator Thunberg), from the genus Opius, e.g. Opius pallipes Wesmael, from the genus Praon, e.g. Praon volucre, from the genus Psyttalia, e.g. Psyttalia concolor Szepligeti;
the family Diapriidae, such as from the genus Trichopria, e.g. Trichopria drosophilae (Perkins);
the family Dryinidae, such as from the genus Neodryinus, e.g. Neodryinus typhlocybae (Ashmead);
the family Encyrtidae, such as from the genus Acarophagus, e.g. Acerophagus maculipennis Mercet, Acerophanus artelles, from the genus Aenasius, e.g. Aenasius arizonensis, from the genus Anagyrus, e.g. Anagyrus dactylopii (Howard), Anagyrus aberiae, Anagyrus fusciventris (Girault), Anagyrus pseudococci (Girault), Anagyrus sinope Noyes & Menezes, Anagyrus vladimiri, from the genus Arrhenophagus, e.g. Arrhenophagus albitibiae Girault, from the genus Blastothrix, e.g. Blastothrix brittanica Girault, from the genus Coccidencyrtus, e.g. Coccidencyrtus ochraceipes Gahan, Coccidoxenoides perminutus Girault, from the genus Comperiella, e.g. Comperiella bifasciata Howard, from the genus Cryptanusia, e.g. Cryptanusia comperei Timberlake, Cryptanusia aureiscutellum (Girault), Genus Dicarnosis, e.g. Dicarnosis ripariensis, from the genus Encyrtus, e.g. Encyrtus aurantii (=lacaniorum) (Geoffroy), Encyrtus infelix (Embleton), from the genus Gyranusoidea, e.g. Gyranusoidea litura Prinsloo, from the genus Leptomastidea, e.g. Leptomastidea abnormis (Girault), from the genus Leptomastix, e.g. Leptomastix algirica Trjapitzin, Leptomastix dactylopii Howard, Leptomastix epona (Walker), Leptomastix histrio (Forster), from the genus Metaphycus, e.g. Metaphycus flavus (Howard), Metaphycus helvolus (Compere), Metaphycus lounsburyi (Howard), Metaphycus stanleyi Compere, Metaphycus swirskii Annecke & Mynhardt, from the genus Microterys, e.g. Microterys nietneri (Motschulsky), from the genus Ooencyrtus, e.g. Ooencyrtus kuwanae (Howard), Ooencyrtus pityocampae (Mercet), from the genus Tetracnemoidea, e.g. Tetracnemoidea brevicornis (Girault), Tetracnemoidea peregrina (=Hungariella peregrina) (Compere), from the genus Tamarixia, e.g. Tamarixia triozae;
the family Eulophidae, such as from the genus Anastatus, e.g. Anastatus japonicus (Ashmead), from the genus Aprostocetus, Aprostocetus hagenowii (Ratzeburg), from the genus Chouioia, e.g. Chouioia cunea Yang, from the genus Diglyphus, e.g. Diglyphus isaea (Walker), from the genus Elasmus, e.g. Elasmus nudus (Nees), from the genus, e.g. Neochrysocharis formosa (Westwood), from the genus Necremnus, e.g. Necremnus artynes (Walker), from the genus Tetrastichus, e.g. Tetrastichus coeruleus (asparagi) (Nees), from the genus Thripobius, e.g. Thripobius javae (Girault) (=semiluteus Boucek);
the family Figitidae, such as from the genus Leptopilina, e.g. Leptopilina heterotoma (Thomson);
the family Mymaridae, such as from the genus Anagrus, e.g. Anagrus atomus (Linnaeus), from the genus Anaphes, Anaphes iole Girault;
the family Platygastridae, such as from the genus Allotropa, e.g. Allotropa convexifrons Muesebeck, Allotropa musae Buhl;
the family Pteromalidae, such as from the genus Anisopteromalus, e.g. Anisopteromalus calandrae (Howard), from the genus Catolaccus, e.g. Catolaccus hunteri, Catolaccus grandis, from the genus Dibrachys, e.g. Dibrachys microgastri (Bouche), from the genus Lariophagus, Lariophagus distinguendus (Forster), from the genus Muscidifurax, e.g. Muscidifurax raptor Girault & Sanders, Muscidifurax raptorellus Kogen & Legner, Muscidifurax zaraptor Kogan & Legner, from the genus, e.g. Nasonia vitripennis (Walker), from the genus Ophelosia, e.g. Ophelosia crawfordi Riley, from the genus Scutellista, e.g. Scutellista caerulea (Fonscolombe), from the genus Spalangia, e.g. Spalangia cameroni Perkins;
the family Scelionidae, such as from the genus Trissolcus, Trisslocus basalis, Trissolcus japonicus, from the genus Telenomus, e.g. Telenomus podisi, Telenomus remus;
the family Trichogrammatidae, such as from the genus Trichogramma, e.g. Trichogramma achaeae Nagaraja & Nagarkatti, Trichogramma brassicae Bezdenko, Trichogramma cacoeciae Marchal, Trichogramma chilonis Ishii, Trichogramma dendrolimi Matsumura, Trichogramma embryophagum (Hartig), Trichogramma euproctidis Girault, Trichogramma evanescens Westwood, Trichogramma galloi, Trichogramma japonicum Ashmead, Trichogramma ostriniae Peng & Chen, Trichogramma pretiosum, Trichogramma pintoi Voegele, from the genus Trichogrammatoidea, e.g. Trichogrammatoidea cryptophlebiae.

Within the order Neuroptera suitable predatory beneficial arthropods may be selected from:
the family Chrysopidae, such as from the genus Chrysopa, e.g. Chrysopa sinica Tiedet, from the genus Chrysoperla, e.g. Chrysoperla carnea Stephens, Chrysoperla lucasina (Lacroix), Chrysoperla rufilabris (Burmeister), from the genus Coniopteryx, e.g. Coniopteryx tineiformis Curtis, from the genus Conwentzia, e.g. Conwentzia psociformis (Curtis), from the genus Micromus, e.g. Micromus angulatus (Stephens), Micromus variegatus, from the genus Sympherobius, e.g. Sympherobius fallax Navas.

Within the order Thysanoptera suitable predatory beneficial arthropods may be selected from:
the family Aeolothripidae, such as from the genus Aeolothrips, e.g. Aeolothrips intermedius Bagnall, from the genus Franklinothrips, e.g. Franklinothrips megalops (Trybom), Franklinothrips vespiformis (Crawford);
the family Phlaeothripidae, such as from the genus Aleurodothrips, e.g. Aleurodothrips fasciapennis (Franklin), from the genus Haplothrips, e.g. Haplothrips brevitubus (Karny), from the genus Karnyothrips, e.g. Karnyothrips melaleucus Bagnall;
the family Thripidae, such as from the genus Scolothrips, e.g. Scolothrips sexmaculatus (Pergande).

Within the order Mesostigmata suitable predatory beneficial arthropods may be selected from:
the family Phytoseiidae such as from:
the subfamily of the Amblyseiinae, such as from the genus Amblyseius, e.g. Amblyseius andersoni, Amblyseius aerialis, Amblyseius swirskii, Amblyseius herbicolus or Amblyseius largoensis, from the genus Euseius e.g. Euseius finlandicus, Euseius hibisci, Euseius ovalis, Euseius victoriensis, Euseius stipulatus, Euseius scutalis, Euseius tularensis, Euseius addoensis, Euseius concordis, Euseius ho, Euseius gallicus, Euseius citrifolius or Euseius citri, from the genus Iphiseiodes e.g. Iphiseiodes zuluagi, from the genus Iphiseius e.g. Iphiseius degenerans, from the genus Neoseiulus e.g. Neoseiulus barkeri, Neoseiulus californicus, Neoseiulus cucumeris, Neoseiulus longispinosus, Neoseiulus womersleyi, Neoseiulus idaeus, Neoseiulus anonymus, Neoseiulus paspalivorus, Neoseiulus reductus or Neoseiulus fallacis, Neoseiulus baraki from the genus Amblydromalus e.g. Amblydromalus limonicus from the genus Typhlodromalus e.g. Typhlodromalus aripo, Typhlodromalus lailae or Typhlodromalus peregrinus from the genus Transeius (alternatively known as Typhlodromips) e.g. Transeius montdorensis (alternatively known as Typhlodromips montdorensis), from the genus Phytoseiulus, e.g. Phytoseiulus persimilis, Phytoseiulus macropilis, Phytoseiulus longipes, Phytoseiulus fragariae;
the subfamily of the Typhlodrominae, such as from the genus Galendromus e.g. Galendromus occidentalis, from the genus Metaseiulus e.g. Metaseiulus flumenis, from the genus Gynaeseiu e.g. Gynaeseius liturivorus from the genus Typhlodromus e.g. Typhlodromus exhilarates, Typhlodromus phialatus, Typhlodromus recki, Typhlodromus transvaalensis, Typhlodromus pyri, Typhlodromus doreenae or Typhlodromus athiasae;
the family Ascidae such as from the genus Proctolaelaps, such as Proctolaelaps pygmaeus (Muller); from the genus Blattisocius e.g. Blattisocius tarsalis (Berlese), Blattisocius keegani (Fox); from the genus Lasioseius e.g. Lasioseius fimetorum Karg, Lasioseius floridensis Berlese, Lasioseius bispinosus Evans, Lasioseius dentatus Fox, Lasioseius scapulatus (Kenett), Lasioseius athiasae Nawar & Nasr; from the genus Arctoseius e.g. Arctoseius semiscissus (Berlese); from the genus Protogamasellus e.g. Protogamasellus dioscorus Manson;
the family Laelapidae such as from the genus Stratiolaelaps e.g. Stratiolaelaps scimitus (Womersley); Gaeolaelaps e.g. Gaeolaelaps aculeifer (Canestrini); Androlaelaps e.g. Androlaelaps casalis (Berlese), Cosmolaelaps e.g. Cosmolaelaps claviger, Cosmolaelaps jaboticabalensis;
the family Macrochelidae such as from the genus Macrocheles e.g. Macrocheles robustulus (Berlese), Macrocheles muscaedomesticae (Scopoli), Macrocheles matrius (Hull);
the family Parasitidae such as from the genus Pergamasus e.g. Pergamasus quisquiliarum Canestrini; Parasitus e.g. Parasitus fimetorum (Berlese), Parasitus bituberosus, Parasitus mycophilus, Parasitus mammilatus;

Within the order Trombidiformes suitable predatory beneficial arthropods may be selected from:
the family Tydeidae such as from the genus Homeopronematus e.g. Homeopronematus anconai (Baker); from the genus Tydeus e.g. Tydeus lambi (Baker), Tydeus caudatus (Duges); from the genus Pronematus e.g. Pronematus ubiquitous (McGregor);
the family Cheyletidae such as from the genus Cheyletus e.g. Cheyletus eruditus (Schrank), Cheyletus malaccensis Oudemans;
the family Cunaxidae such as from the genus Coleoscirus e.g. Coleoscirus simplex (Ewing), from the genus Cunaxa e.g. Cunaxa setirostris (Hermann);
the family Erythraeidae such as from the genus Balaustium e.g. Balaustium putmani Smiley, Balaustium medicagoense Meyer &Ryke, Balaustium murorum (Hermann), Balaustium hernandezi, Balaustium leanderi;
   the family Stigmaeidae such as from the genus Agistemus e.g. Agistemus exsertus Gonzalez, from the genus Zetzellia, e.g. Zetzellia mali (Ewing);
the family Anystidae, such as from the genus Anystis, e.g. Anystis baccarum.

In view of their predatory behaviour towards important pests, predatory mites preferably are selected from the family Phytoseiidae, in particular from the genus Amblyseius, such as Amblyseius swirskii, Amblyseius largoensis and Amblyseius andersoni, from the genus Neoseiulus, such as Neoseiulus californicus, Neoseiulus cucumeris, Neoseiulus barkeri, Neoseiulus baraki and Neoseiulus longispinosus and Neoseiulus fallacis, in particular from the genus Euseius, such as Euseius gallicus, in from the genus Iphiseius, such as Iphiseius degenerans, from the genus Transeius, such as Transeius montdorensis, from the genus Amblydromalus, such as Amblydromalus limonicus (alternatively known as Typhlodromalus limonicus), from the genus Galendromus, such as Galendromus occidentalis, from the genus Phytoseiulus, such as Phytoseiulus persimilis, Phytoseiulus macropilis and Phytoseiulus longipes, from the family Cheyletidae, in particular from the genus Cheyletus, such as Cheyletus eruditus, from the family Laelapidae, in particular from the genus Androlaelaps, such as Androlaelaps casalis, from the genus Stratiolaelaps, such as Stratiolaelaps scimitus (Alterntively known as Hypoaspis miles), from the genus Gaeolaelaps, such as Gaeolaelaps aculeifer (Alternatively known as Hypoaspis aculeifer), or from the family Macrochelidae, in particular from the genus Macrocheles, such as Macrocheles robustulus.

Beneficial arthropods that may serve as a prey for predatory arthropods may be selected from the Astigmata, in particular from:
the family Carpoglyphidae such as from the genus Carpolyphus e.g. Carpoglyphus lactis;
the family Pyroglyphidae such as from the genus Dermatophagoides e.g. Dermatophagoides pteronysinus, Dermatophagoides farinae; from the genus Euroglyphus e.g. Euroglyphus longior, Euroglyphus maynei; from the genus Pyroglyphus e.g. Pyroglyphus africanus;
the family Glycyphagidae such as from the subfamily Ctenoglyphinae, such as from the genus Diamesoglyphus e.g. Diamesoglyphus intermediusor from the genus Ctenoglyphus, e.g. Ctenoglyphus plumiger, Ctenoglyphus canestrinii, Ctenoglyphus palmifer; the subfamily Glycyphaginae, such as from the genus Blomia, e.g. Blomia freemani or from the genus Glycyphagus, e.g. Glycyphagus ornatus, Glycyphagus bicaudatus, Glycyphagus privatus, Glvcvphagus domesticus, or from the genus Lepidoglyvphus e.g. Lepidoglyphus michaeli, Lepidoglyphus fustifer, Lepidoglyphus destructor, or from the genus Austroglycyphagus, e.g. Austroglycyphagus geniculatus; from the subfamily Aeroglyphinae, such as from the genus Aeroglyphus, e.g. Aeroglyphus robustus; from the subfamily Labidophorinae, such as from the genus Gohieria, e.g. Gohieria fusca; or from the subfamily Nycteriglyphinae such as from the genus Coproglyphus, e.g. Coproglyphus stammeri or from the subfamily Chortoglyphidae, such as the genus Chortoglyphus e.g. Chortoglyphus arcuatus and more preferably is selected from the subfamily Glycyphaginae;
the family Acaridae such as from the genus Tyrophagus e.g. Tyrophagus putrescentiae, Tyrophagus tropicus, from the genus Acarus e.g. Acarus siro, Acarus farris, Acarus gracilis; from the genus Lardoglyphus e.g. Lardoglyphus konoi, from the genus Thvreophaus, such as Thyreophagus entomophagus; from the genus Aleurogvphus, e.g. Aleuroglyphus ovatus;
the family Suidasiidae such as from the genus Suidasia, such as Suidasia nesbiti, Suidasia pontifica or Suidasia medanensis.

Preferred Astigmatid mites may be selected from the family Glycyphagidae, such as from the genus Lepidoglyphus, e.g. Lepidoglyphus destructor, the family Carpoglyphidae such as from the genus Carpoglyphus e.g. Carpoglyphus lactis, the genus Thyreophagus, such as Thyreophagus entomophagus, Acaridae, such as Suidasia pontifica or Suidasia medanensis.

Astigmatid mites can be isolated from their natural habitats as described by Hughes (Hughes, A. M., 1977, The mites of stored food and houses. Ministry of Agriculture, Fisheries and Food, Technical Bulletin No. 9: 400 pp), and can be maintained and cultured as described by Parkinson (Parkinson, C. L., 1992, “Culturing free living astigmatid mites.” Arachnida: Proceedings of a one day symposium on spiders and their allies held on Saturday 21st November 1987 at the Zoological Society of London) and by Solomon & Cunnington (Solomon, M. E. and Cunnington, A. M., 1963, Rearing acaroidmites, Agricultural Research Council, Pest Infestation Laboratory, Slough, England, pp 399 403).

Selection of the beneficial arthropods is most preferably made from the family Anthocoridae and Miridae, but the present invention will also have benefits for beneficial arthropods from the other families presented. An overview of the many biological control agents used is presented in Mason P. (ed.) Biological control. Global impacts, challenges and future directions of pest management. CRC Press (2021).

The system for releasing beneficial arthropods comprises a housing, the arthropod housing, suitable to house the beneficial arthropods. The housing comprises a body, preferably the body comprises a plate form. However, housings with a body having a different form or shape may be used. The body is provided with a number of chambers which provide an accessible interior space for the arthropods. The essential function of the body is to accommodate the number of chambers. The number of chambers preferably is a plurality. Within the context of the present invention “a plurality” should be understood to mean two or more, such as 2, 3, 4, 5, 6, 7, 8, 9, 10. When the body comprises a plate form, it is preferred that the plate form is a flat plate. However, the use of bend plates is also envisaged within the present invention. When bodies comprising a plate form are used, it is further preferred that the arthropod release system comprises different plate parts or plates, connected by a hinging element that makes it possible for the hinging plate parts or plates to move relative to each other. The hinging element preferably is suitable to allow the hinging plate parts or plates to fold together, in particular in a number of V-shapes. The V-shaped structure, when reversed (upside down) may be used to hang the arthropod distribution system on a thread or a branch or a similar element in the target area. Alternatively, arthropod distribution systems can comprise other means for hanging, such as a number of hooks or threads. Such hanging means may be fixed to the body of the arthropod distribution system.

The skilled person will understand that shapes and forms other than plates are suitable to accommodate the chambers. For example, the body may comprise a block having a rectangular shape, such as a cubical, pyramidal, trapezoidal or other rectangular shape. A spherical shape may also be possible.

The shape of the interior space of the number of chambers preferably is such that the dimensions in at least one direction are smaller than the dimensions in another direction. The directions referred to are the three perpendicular directions x-y-z of an (imaginary) three-dimensional Cartesian coordinate system. The skilled person will understand that when a chamber has a shape for which the dimensions in one direction are smaller than, such as half of, the dimensions in one of the other directions, the chamber will have a “low”, “narrow” or “short” shape when the shortest dimension would be in the direction of the “height”, “width” or “length” respectively. According to a preferred embodiment the dimensions in the smallest direction are <0.5, such as <0.4, <0.3, <0.2, <0.1, of the dimensions in the biggest direction. The lower limit of the dimensions is determined by the size of the arthropods, it will be clear that the arthropods should fit within the chambers. Preferably the volume of the chambers is at least 3 times, such as at least 4, 5, 6, 7, 8, 9, 10 times the volume of the arthropods. The volume V of the arthropods referred to is the volume measured by approximation by V=W²×L, wherein W is the average width of the bodies of the arthropod individuals and L is the average length of the bodies of the arthropod individuals. Values for average width (W) and length (L) can readily be determined by the skilled person, for a number of preferential arthropods the values for W and L considered within the context of this invention are listed in Table 1 below. If for a specific arthropod species, a range is presented for the average length and width, the highest values should be considered for determining the preferred minimal volume of the chambers.

TABLE 1
Values for average width of the bodies of the arthropod individuals
(W) and the average length of the bodies of the arthropod individuals
(L) of exemplary beneficial arthropods preferred in the invention.

| | Average | Average |
|---|---|---|
| | length | width |
| BENEFICIAL ARTHROPOD | (mm) | (mm) |
| Family Coccinellidae | | |
| Cryptolaemus montrouzieri Mulsant | 3.5-4.5 | 1.8-2.3 |
| Delphastus catalinae Horn (syn. Delphastus pusillus | 1.3-1.5 | 0.7-0.8 |
| LeConte)) | | |
| Family Anthocoridae | | |
| Orius insidiosus (Say) | 2.0-2.5 | 0.7-0.8 |
| Orius laevigatus (Fieber) | 1.4-2.4 | 0.6-0.8 |
| Orius majusculus | 1.8-2.2 | 0.6-0.7 |
| Orius strigicollis (Poppius) | 1.6-2.1 | 0.6-0.8 |
| Family Lygaidea | | |
| Geocoris punctipes | 3.5-4.0 | 1.2-1.3 |
| Family Miridae | | |
| Macrolophus pygmaeus (Rambur) | 3.0-3.6 | 0.8-0.9 |
| Nesidiocoris tenuis (Reuter) | 3.0-4.0 | 0.8-1.0 |
| Family Aphelinidae | | |
| Aphytis melinus DeBach | 1.0 | 0.3 |
| Family Braconidae | | |
| Dacnusa sibirica Telenga | 2.0-3.0 | 0.4-0.5 |
| Family Eulophidae | | |
| Diglyphus isaea (Walker) | 2.0-3.0 | 0.4-0.5 |
| Family Cheyletidae | | |
| Cheyletus eruditus (Schrank) | 0.5 | 0.3 |
| Family Laelapidae | | |
| Androlaelaps casalis (Berlese) | 0.7 | 0.5 |
| Stratiolaelaps scimitus (Womersley) | 1.0 | 0.7 |
| Family Macrochelidae | | |
| Macrocheles robustulus (Berlese) | 0.8 | 0.5 |
| Famliy Phytoseiidae | | |
| Amblydromalus limonicus (Garman & McGregor) | 0.4 | 0.3 |
| Amblyseius andersoni (Chant) | 0.4 | 0.3 |
| Amblyseius swirskii (Athias-Henriot) | 0.4 | 0.3 |
| Neoseiulus californicus (McGregor) | 0.4 | 0.3 |
| Neoseiulus cucumeris (Oudemans) | 0.4 | 0.3 |
| Phytoseiulus persimilis Athias-Henriot | 0.5 | 0.3 |
| Transeius montdorensis (Schicha) | 0.4 | 0.3 |
| Family Acaridae | | |
| Tyrophagus putrescentiae | 0.3 | 0.2 |
| Family Carpoglyphidae | | |
| Carpoglyphus lactis | 0.5 | 0.3 |
| Family Glycyphagidae | | |
| Lepidoglyphus destructor | 0.4 | 0.3 |

The chambers preferably are elongated in shape. As the skilled person will understand in general and from the further description of the present invention and the attached figures, the shape of a chamber is elongated when the dimension in two directions (for example the direction along the (imaginary) x and y axes) is substantially smaller than the dimensions in the other perpendicular direction (for example the directions along the (imaginary) z axis). The skilled person will also understand that an elongated shape is not limited to straight and rectangular shapes and that elongated chambers may also be bend and/or comprise corners. The use of bend elongated chambers for the arthropods explicitly is part of the present invention. The shape of the cross section of elongated chambers may vary. It is however preferred that the shape used provides an even surface for the arthropod individuals. For example, circular, rectangular, triangular shapes and derived shapes may be used. In many cases elongated chambers have a first and a second end. However, a ring or O-shaped chamber may also be considered an elongated chamber, but in this case having a bend shape. Chambers comprising a ring or O-shape or having a similar and/or derived shape, such as a spiral (helix) shape are also within the present invention. The skilled person will understand that a ring or O-shaped chamber does not have a first and second end.

For access of the arthropods to the chambers, there is a number of openings in the chambers. The skilled person will understand that the openings can be accessed from outside of the body of the arthropod housing. Within the context of the present invention “a number of” should be understood to mean one or more, such as a plurality. When a number of chambers, such as all chambers, have a first and second end, an opening is preferably provided on at least one end, more preferable an opening is provided at both the first and the second end. The dimensions of the openings are such that the arthropods can access the interior space of the number of chambers. It is preferred that the ratio between the diameter of the openings and the average width of the bodies of the selected arthropods is 1.5-15, more preferably 1.5-7.5, such as 2.0-7.5, most preferably 2.5-4.5. Values for average width of the bodies of the arthropod individuals are known or can be readily determined by the skilled person. Table 1 above presents values for a number of preferred beneficial arthropods. When a chamber has an elongated shape, it is preferred that an opening is located on at least one end of the chamber and that the opening has a size and shape corresponding to the size and shape of the cross section of the chamber.

According to a preferred embodiment the body of the arthropod housing has a plate form, the chambers have an elongated shape and the axis of the number of chambers are essentially perpendicular to the normal vector of the plane of the body. The axis of the number of chambers referred to is the longest axis running in the direction of the elongation of the elongated chambers. In this situation the chambers preferably run through the plane of the body, but they can also run on the surface of the plate formed body. It is most preferred that the body of the arthropod housing has the form of a flat plate, but it is possible that the plate has a bend shape. It is further preferred that the number of chambers are a plurality of essentially straight chambers running essentially parallel. The skilled person will understand, that for a bend plate the direction of the normal vector will be different at different positions on the bend plate, thus the direction of the axis of the chamber will also change and the chamber will also have a bend shape.

The chambers of the arthropod housing comprise individuals of the beneficial arthropod species. An arthropod housing containing in its chambers the individuals of the beneficial arthropod species forms the system for releasing beneficial arthropods. Such a system thus comprises a structural element (the arthropod housings) and biological elements (the arthropod individuals). It is considered more appropriate to refer to them with the term system, however, they can also be referred to as a device. Thus, whenever in the context of this invention the term “system for releasing beneficial arthropods” or its plural form is used, this may be replaced by the term “device for releasing beneficial arthropods” or its plural form. The individuals preferably form a breeding population of the beneficial arthropod species. In this specification the term “breeding” must be understood to include the propagation and increase of a population by means of reproduction. The skilled person will know and understand that although many beneficial arthropod species reproduce via sexual reproduction, some species reproduce via asexual reproduction. The skilled person will be able to identify which arthropod species reproduce sexually and which arthropod species reproduce asexually. In essence a breeding population is capable of increasing the number of its individuals by means of reproduction. The skilled person will thus understand that a breeding population will comprise female individuals that are capable to reproduce, i.e. that can produce off spring, or female individuals that can mature to a life stage wherein they can produce offspring. The skilled person will further understand that for an arthropod species that reproduces sexually a breeding population also comprises sexually mature male individuals or male individuals that may mature to sexually mature male individuals. Alternatively for an arthropod species reproducing sexually a breeding population may comprise one or more fertilized females.

The individuals of the arthropod species further preferably are motile individuals. The skilled person will understand that motile individuals are individuals that have the capability of moving from a first location to a second location spontaneously and independently. For the different beneficial arthropods presented above that may be selected within the present invention, the skilled person will know and understand which life stages are motile. For example, for parasitic wasps it is known that only the adult stages have motility. Eggs larva and pupae are known not to have motility, but are confined to the body of their host. Eggs larva and pupae for parasitic wasps are thus not considered motile life stages within the context of the present invention. Other Predatory insects used in the present invention may be holometabolous or hemimetabolous insects. For holometabolous insects, in general the larval or nymphal and imago (adult) stages have motility, whereas the egg and pupa stages do not. For hemimetabolous insects in general the nymph and imago stage have motility, whereas the egg does not. As the skilled person knows, life stages of predatory mites and Astigmatid prey mites which are motile are larvae, nymphs and adults, while the egg stage is not motile. Within the present invention it is most preferred that the chambers contain adult life stages, most preferably of both sexes, of the selected beneficial arthropod.

According to a preferred embodiment chambers contain a plurality of beneficial arthropod individuals. It should be emphasized that for this embodiment it is not required that all chambers contain a plurality of individuals, but that it is sufficient that the majority (>51%) of the chambers contain a plurality of individuals.

When the chambers are elongated chambers, it is preferred that the beneficial arthropods may move freely in the chambers, such that individuals may change their direction of movement in the chambers and/or such that individuals may pass each other in the chambers. Free movement of the arthropod individuals within the chambers of a single arthropod housing improves distribution of the arthropod individuals between arthropod housings, when a plurality of housings is used. Therefore, it is preferred that when the average length of the bodies of individuals of the selected beneficial arthropod species is L and the diameter of the elongated chambers of the arthropod housing is D the ratio D/L is >1.1, such as >1.2, >1.5, preferably >2.5. Too much free space around the arthropods in the chambers may also not be optimal, as aspects of thigmotaxis may also play a role in the arthropods behavior to reside in the chambers. The ratio D/L may be between 1.1 and 6.0, most preferably between 2.5 and 3.5. According to an alternative embodiment it is preferred that when the average width of the bodies of individuals of the selected beneficial arthropod species is W and the diameter of the elongated chambers of the arthropod housing is D, the ratio D/W is >1.2, preferably >2.5. The ratio D/W may be between 1.2-15, more preferably 1.5-7.5, such as 2.5-7.5, most preferably 2.5-4.5. Values for average width of the bodies of the arthropod individuals (W) and the average length of the bodies of the arthropod individuals (L) of exemplary beneficial arthropods preferred in the invention are presented in table 1 above.

According to a further aspect the present invention relates to a system for storing beneficial arthropods. Whenever in the context of this invention the term “system for storing beneficial arthropods” or its plural form is used, this may be replaced by the term “device for storing beneficial arthropods” or its plural form. Storing according to certain embodiments should be understood to mean to keep a substantial fraction of the beneficial arthropods alive while confined over a period of time. The storing could for example be in a storage after production, at facilities where the arthropods are to be used in pest control in the target area or during transport from the production facility to the facilities where they are used in pest control. As the skilled person will understand it is normal that over time individuals of a population of beneficial arthropods may die due to different causes, such as their age, cannibalism, food and/or water starvation (due to an individual's incapacity to reach sufficient food or water) etcetera. Thus, deaths of beneficial arthropods will regularly happen during their storage (including transport) due to these natural reasons. However, deaths may significantly increase due to inadequate methods of storage. The system for storing beneficial arthropods of the present invention does not and cannot prevent all deaths of beneficial arthropods during storage, instead the aim is to efficiently provide a storage solution having acceptable storage losses. After storage, the system for storing beneficial arthropods and in particular certain of its parts can be used for releasing the beneficial arthropods.

The system for storing beneficial arthropods of the invention comprises a container having a base and a number of walls extending from the base. The number of walls surround an open space or a void. The base and the number of walls need not be distinguishing structural elements but could be part of a continuous plane shaped to provide a base section and a number of wall sections, similar to for example a bathtub. The base and the number of walls surround a void (open space). In the void a number of arthropod release systems according to the invention are positioned. It is preferred that a plurality of arthropod release systems is provided in the void. The shape of the void preferably is optimized for holding the number of arthropod release systems, depending on the shape, number and relative positioning of the arthropod release systems.

The arthropod releasing system of the invention thus is an essential element of the system for storing beneficial arthropod. These two systems are interrelated products, as the system for releasing the arthropods can be considered an intermediate product, which provides an important contribution to the effectiveness of the total system.

According to a preferred embodiment a plurality of arthropod release systems is positioned in the void, such that openings of the chambers are facing the opening of the void. This makes it possible for arthropods which are placed on top of the openings to access the chambers. It is preferred that openings of chambers of arthropod housings of all arthropod release systems face the opening of the void. It is further preferred in this embodiment that the plurality of arthropod release systems has a tight fitting in the void. Due to the tight fitting of the plurality of arthropod release systems there remains little free space in the void where the arthropod individuals could reside. This prevents that arthropod individuals could reside in spaces between the bodies of the arthropod release systems, where they could become injured if the release systems would move relative to each other. In addition, this reduces the space in the void outside the chambers of the arthropod release systems, and thus increases the chance that arthropod individuals will reside in the chambers of the arthropod release systems. According to an embodiment of the preferred embodiment, a plurality of arthropod release systems having plate shaped bodies is provided in the void, wherein the plates are stacked. The stacking of plate shaped bodies preferably has a tight fitting in the void, as explained. It is further preferred for this embodiment that the arthropod release systems comprise a number of plates, preferably a plurality, connected by a hinging element that makes it possible for the hinging parts of a plate, or of hinging plates, to move relative to each other.

According to a preferred embodiment, a tight fitting of a number of, preferably a plurality, of arthropod release systems when positioned in the void may be achieved in that the total surface facing the opening of the void of the arthropod housings of the number of release systems, is 0.80-0.97, preferably 0.87-0.95, of the surface of the cross section of the opening of the void, in particular the surface of the cross section of the void where the surface of the arthropod housings is located.

According to a preferred embodiment a food source for the individuals of the beneficial arthropod species is also provided in the void. This food source may be provided in the void unconnected to the arthropod releasing system, or may be in association with the arthropod releasing system such as connected to the surface of the body or present in a number of chambers. The skilled person will be able to select a suitable food source for the selected beneficial arthropod present in the chambers of the arthropod releasing system. The skilled person will be able to select suitable food sources for selected life stages of selected beneficial arthropods. For example, depending on the selected life stages of selected beneficial arthropods Artemia, Ephestia eggs, (Astigmatid) prey mites, artificial diets and sugar solutions may be used.

Preferably a water source suitable for the individuals of the beneficial arthropod population is provided in the void. The skilled person will be able to select suitable water sources for selected beneficial arthropods. For example, the skilled person will understand that for certain beneficial arthropods it is sufficient that an adequate moisture level in their environment is sufficient. Adequate moisture levels may be selected by the skilled person. In general, 80-85% RH may be considered most favorable, but some deviation may be acceptable, such as 60-90% RH. Maintaining adequate moist levels can for example be supported by providing a material that is capable of releasing water vapor. Water vapor may for example be released by a mass of water bound by a gelling agent, such as a gelling agent selected from agar-agar, gellan gum, xanthan gum, guar gum or a different vegetable gum. The use of a microbiological inert gelling agent is preferred in order to prevent that microbial growth is stimulated. The gel of water bound by the gelling agent may be formed by using the gelling agent in an amount known to the skilled person, such as 1-5% (w/w), preferably 1-3%. Alternatively, water vapor may be released from a mass of water bound in a (micro) fiber matrix, such as from a material comprising plant fibers, such as cellulose fibers. Absorption paper or cotton materials could for example be used to bind a mass of water. A soluble food source, such as sugar may be dissolved in the water, to provide a food source for the beneficial arthropods.

Optionally the water source is provided together with a separation means, suitable to separate the water source in a separated part of the void. The separation means prevents direct contact of water with other elements in the void, such as the number of arthropod housings, which reduces the chance that they may drown. The separation means may also reduce and preferably prevents direct access of the arthropods to the water source. Reducing the possibility of direct access of the arthropods with the water source reduces the chance that arthropods remain in the vicinity of the water source and do not enter the chambers. When the water source is for example a material that is capable of releasing water vapor, such as a mass of water bound by a gelling agent or in a (micro) fiber matrix, it could be provided on the bottom of the base. The separation means could be a floor plate lifted some distance from the base, for example by resting on a number of spacing elements protruding from the base or on a ridge connected to or incorporated in the number of walls. While preventing direct access of the arthropod individuals with the water source, the separation means preferably does allow transfer of water vapor, such that the humidity in the part of the void where the arthropods reside is maintained within desired levels, at least as much as possible. Simple means as cardboard plates can be used as separation means.

The system for storing arthropods comprises closing means which close of the interior space of the void from the exterior space. Closing means may be selected from a lid or a sheet, preferably providing a tight closing. It will be clear for the skilled person that the void should be closed off to the extent that escape of the arthropods is minimized and preferably prevented. Furthermore, it is preferred that the void is closed off with a covering, allows some exchange of metabolic gasses, to avoid the risk of suffocation of the arthropods, especially during longer storage periods. Closing coverings made from sheet paper can be suitably used as the closing means. Depending on the phototaxis behavior of the selected beneficial arthropod and the desire to influence this positively or negatively, e.g. to influence their behavior to enter and remain in the chambers, the closing means may be transparent, partially transparent or non-transparent.

For providing a tight closing, the closing means preferably has a form corresponding to the form of the edge(s) of the number of walls surrounding the voids. For providing a tight closing, the closing means may have elements that cooperate with elements on the number of walls, such as snap closure elements. Alternatively, tight closure of the closing means may be accomplished by sealing it on the number of walls, preferably on the edge(s) of the number of walls. The seal connection may be achieved by any known means, such as by using a separate adhesive and/or a heat-sealing technique. For certain embodiments, the use of a flexible closing means, such as a closing foil or sheet paper is preferred. When a foil or sheet paper is used, closing preferably is achieved by sealing, such as heat sealing.

After storage, including storage during transport, the system for storing beneficial arthropods can be used for releasing the beneficial arthropods in a target area. For this the closure of the void may be opened near to or in the target area. After opening, a number of arthropod distribution systems are taken out of the void and placed in the target area. This allows the beneficial arthropods, present in the number of chambers, to exit from the chambers and enter the target area. Preferably all arthropod distribution systems present in the void are placed in the target area. The number of arthropod distribution system may be placed in any suitable way in the target area, for example by hanging it in the target area. If a arthropod distribution system comprise a plurality of plates connected by a hinging element, the hinging element may be suitable for hanging. Alternatively, arthropod distribution systems can comprise other means for hanging, such as a number of hooks or threads. Such hanging means may be fixed to the body of the arthropod distribution system. Part of the beneficial arthropods present in the void may not be present in the chambers, due to the fact that they have not entered a chamber or have exited from a chamber. These arthropods outside of the chambers may be distributed in the target area by shaking them out of the void into the target area. When individuals of the arthropods present in the void have flying capability, they may fly out of the void, when they are not present in a chamber.

The target area may be any area where the activity of the beneficial arthropods is desired. As already discussed, the beneficial arthropods may be predatory or parasitic arthropods or may be mites suitable as a food source for predatory arthropods. An overview of the uses of various biological control agents is presented in Mason P. (ed.) Biological control. Global impacts, challenges and future directions of pest management. CRC Press (2021).

In case the beneficial predatory or parasitic arthropod have a function in controlling a crop pest, the target area may be a crop. The crop may be selected from, but are not restricted to greenhouse or open field vegetable crops such as tomatoes (Solanum lycopersicum), peppers (Capsicum annuum), eggplants (Solanum melogena) Curcubits (Cucurbitaceae) such as cucumbers (Cucumis sativa), melons (Cucumis melo) watermelons (Citrullus lanatus); soft fruit (such as strawberries (Fragaria×annanassa), raspberries (Rubus ideaus)), blueberries, (greenhouse) ornamental crops (such as roses, gerberas, chrysanthemums), tree crops such as Citrus spp. or medicinal crops, such as cannabis.

Prey mites, such as Astigmatid mites, suitable as a food source for predatory arthropods having a function in controlling crop pests may also be released in a crop in order to support the population development of predatory arthropod species present in the crop. Predatory arthropods that may feed on such prey mites may be selected from Mesostigmatid or Prostigmatid predatory mites or from the family Miridae, such as Macrolophus spp., from the family Anthocoridae, such as Orius spp., for example Orius laevigatus, from the family Coccinellidae, such as Adalia spp. or Cryptolaemus montrouzieri, from the Chrysopidae, such as Chrysoperla spp., for example Chrysoperla carnea or from the Lygaeidae, such as Geocoris spp.

According to alternative embodiments, the beneficial arthropods may have a function in controlling pests of an animal, the host animal, in particular pests of domestic animals, including farm animals and companion animals, such as poultry, cattle, horses, dogs or cats. According to such embodiments the target area may be a stable or sleeping area for the host animal. The system according to the invention may for example be used in support of the control of poultry red mite, by comprising as the beneficial arthropod a predatory mites selected from the genus Hypoaspis, such as Hypoaspis angusta, from the genus Cheyletus, such as Cheyletus eruditis, from the genus Androlaelaps, such as Androlaelaps casalis, from the family Laelapidae such as from the genus Stratiolaelaps e.g. Stratiolaelaps scimitus (Womersley); Gaeolaelaps e.g. Gaeolaelaps aculeifer (Canestrini); Androlaelaps e.g. Androlaelaps casalis (Berlese), or from the genus Macrocheles, such as Macrocheles robustulus or an Astigmatid prey mite suitable as prey for a predatory mite from this selection. As the skilled person knows, these predatory mites have broader host ranges and thus may also be employed for controlling other pests. In addition, other beneficial predatory arthropods may also be used to control pests of farm and companion animals. For example, Macrocheles muscadomesticae, Ophyra aenescens and Muscidifurax raptorellus may be used to control flies in stables. The system of the invention may be used to release Astigmatid mites that may serve as a food source for such beneficial predatory arthropods and thus may support the survival and/or development of their populations, thus supporting the control of the pest of the animal host.

In yet other embodiments the beneficial arthropods are predators for pests of stored food products, such as stored product mites. In such embodiments the target area is a food product storage. For example, Bracon hebetor and Cheyletus eruditis may be used for this.

A further aspect of the invention relates to a method for producing the system for storing and/or releasing beneficial arthropods. The features of the different elements of the system for storing beneficial arthropods and the system for releasing beneficial arthropods have already been discussed above and apply also to the method for their production. Thus, they will not be repeated in detail in this part of the discussion of the invention.

In the method in a first step a container is provided. The container has a base and a number of walls extending from the base, said walls surrounding a void.

In addition, a number of arthropod housings of the arthropod distribution system of the invention is provided.

Further, individuals, preferably motile individuals, of a population of the beneficial arthropod are provided. The individuals optionally are on a carrier material. The skilled person will be able to select suitable carrier materials for the individuals of the selected arthropod species. For example, the carrier material may be selected from wood chips, paper pieces, cardboard pieces, husks from plant species, such as buckwheat or Poaceae husks, for example millet husks. As is discussed above, it is preferred that the arthropod individuals that end up in the chambers are motile life stages. These motile life stages will move at own motion into the chambers. It is not required that to achieve this the arthropods provided and positioned in the void are motile life stages, as motile life stages could develop from non-motile life stages, such as eggs or pupae, during the time of storage.

Preferably a food source for the individuals of the beneficial arthropod species is also provided and this is placed in the void.

A water source suitable for the individuals of the beneficial arthropod population may preferably also be provided and placed in the void. Optionally the water source is provided and placed in the void together with a separation means, suitable to separate the water source in a separated part of the void. The separation means preferably is a floor plate, most preferably floor plate that allows transmission of water vapor.

The number of arthropod housing devices and the individuals of the beneficial arthropod population, optionally on a carrier material, are placed in the void. The position of the openings of the chambers preferably is such that they are facing towards the opening of the void, such that the chambers are accessible for the arthropods, when placed in the void on the release systems.

In the method a closing means suitable to close the void is provided and when all required elements are correctly positioned in the void, the void is closed with said closing means.

The steps of the method for producing the system for storing beneficial arthropods need not be performed in the extract order wherein they are discussed above. In fact, any suitable order may be used. For example, the food source, when provided, may be provided and placed in the void after the water source. It is however preferred that as much as possible elements of the system for storing and/or releasing beneficial arthropods are placed in the void in their correct position, before the beneficial arthropods are placed in the void and the void is closed. This allows the time between introduction of the arthropods in the void and closing of the void to be as short as possible, which reduces the risks of escape of arthropod individuals.

From the description above, it will be clear that the system for releasing beneficial arthropods is formed while (and after) performing the method for producing the system for storing beneficial arthropods. This due to the fact that in the method for producing the system for storing beneficial arthropods, the arthropod housing is provided together with the arthropods in such a way that the arthropods are able to access the chambers, which results in the system for releasing beneficial arthropods.

The invention will now further be described with reference to the following figures and experiments, which relate to certain preferred embodiments. It should be understood that the invention is not limited to these specific embodiments of the figures and experiments.

The figures and the description of the invention below relating to these figures are primarily aimed at describing the method of the invention for producing a system for storing and/or releasing beneficial arthropods and the use of the produced system. However, in describing these aspect of the invention, the other aspects of the invention including the system for storing beneficial arthropods and the system for releasing beneficial arthropods are also described.

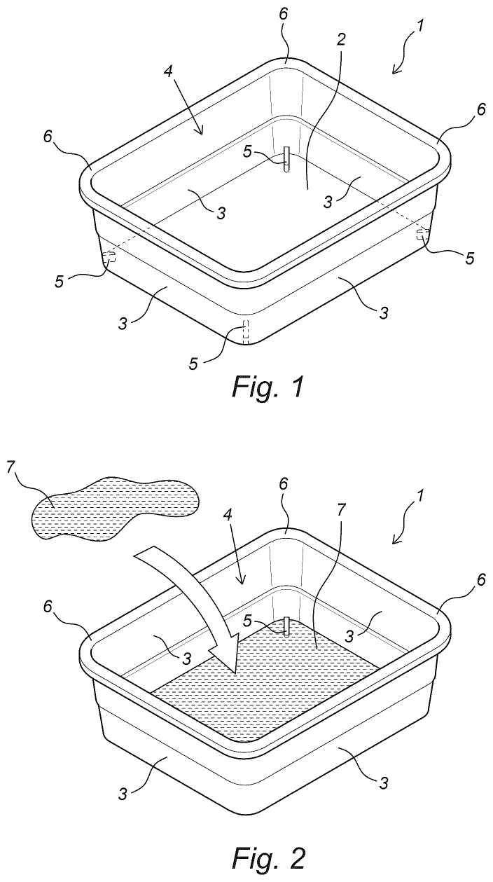

In the method for producing a system for storing and/or releasing beneficial arthropods a container is provided. FIG. 1 presents a perspective view of a container (1) of the system for storing beneficial arthropods as this is provided in an embodiment of the method of the invention. The container (1) having a base (2) and four walls (3) extending from the base (2). The walls (3) with their inner sides surround a void (4). The container (1) of this embodiment is from a renewable material (Earthcycle™, CKF Inc., Langley, Canada) made from mold pressed wood fiber pulp. The base (2) and the walls (3) are from a single continuous bend plane. The container (1) of this embodiment further comprises spacing elements (5) and a closing ridge (6) that will be further discussed below.

In this embodiment of the method an amount of 1% (w/w) agar-agar (7) is provided as a water source for the arthropods, as is shown in FIG. 2. The agar-agar (7) is provided in molten form and poured onto the base (2) of the container (1) and allowed to solidify. The amount of agar-agar (7) poured on the base (2) is adjusted such that its level stays below the top of the spacing elements (5).

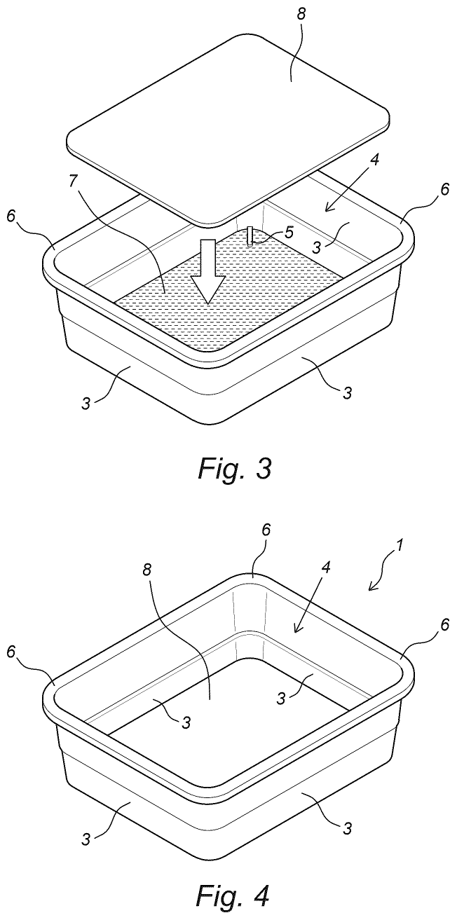

Turning to FIG. 3, it is shown that a floor plate (8) is provided as a separation means, to separate the agar-agar (7) water source positioned on the base (2) from the rest of the void (4). For this the floor plate (8) is positioned in the void, substantially parallel to the base (2), resting on the spacing elements (5), as is shown in FIG. 4. In this embodiment the floor plate is from cardboard and is selected such that water vapor can pass through the plate. The shape of the floor plate (8) is adjusted to the shape of the circumference of the walls (3) at the point of the top of the spacing elements (5) to provide a good fitting of the floor plate (8) in the void (4), such that access of the arthropods to the separated part of the void (4) containing the agar-agar (7) water source is minimized and preferably prevented. In certain embodiments of the invention, additional measures can be taken to further reduce the accessibility of the water source. For example, a mesh which allows passage of moist, but not of arthropods may be positioned (and secured) in the space between the floor plate (8) and the walls (3).

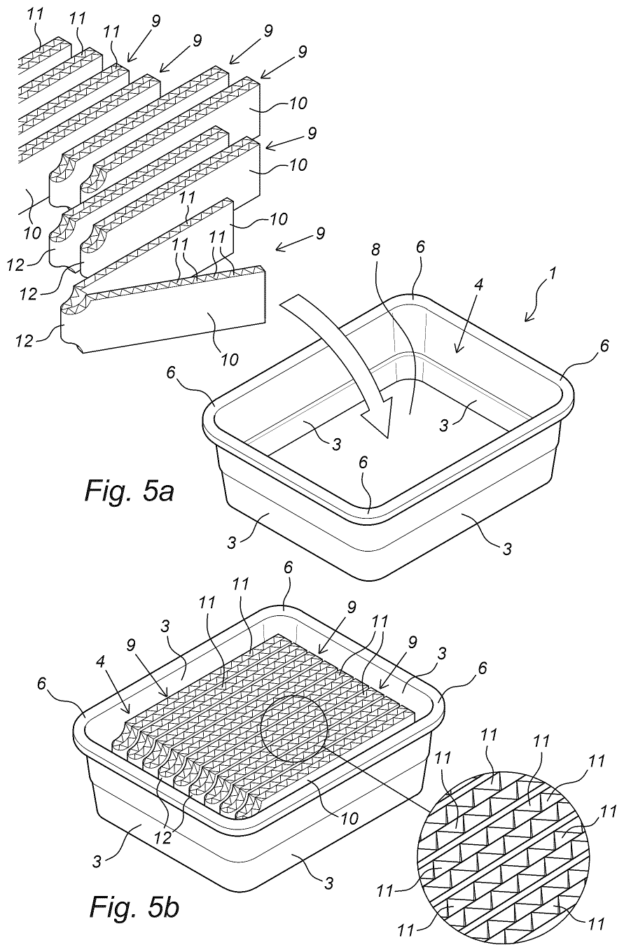

Turning to FIG. 5, it is shown that a number of arthropod housings (9), is provided. The inventors have found that corrugated cardboard has excellent properties and dimensions to serve as arthropod housing in the invention. For the embodiment shown in the figures nine arthropod housings are provided which contain a plate shaped corrugated carboard body (10) having elongated chambers (11). The elongated chambers (11) have a diameter adapted to the size of the beneficial arthropods that are selected to be stored (and distributed) with the system, such that the arthropods are able to access the chambers (11) and preferably can move within the chambers (11) such that they can change their direction of movement and/or that different individuals can pass each other within the chambers (11). It has been found that this improves the movement of the arthropods between the chambers (11), which aids in improving the distribution of the arthropods between different arthropod housings (9). Table 1 above presents suitable size ranges for exemplary beneficial arthropods that may be selected in the present invention. The plate shaped body (10) contains a hinging element (12) formed by indents in the sides of the corrugated cardboard plate (10), which make bending of the material easier. These recesses are not required, as the carboard is pliable and may be bend around a fold. In the present embodiment, the carboard body (10) hinges at the fold of hinging element (12) such that the two plate parts separated by the hinging element (12) are movable to a V-shape, which can be completely closed by bringing the legs together. The nine arthropod housings (9) are placed in the void (4) as a stacking of closed V's. The stacking fits between the walls (3) without leaving much free space around them in the void (4), such that their movement within the void is restricted.

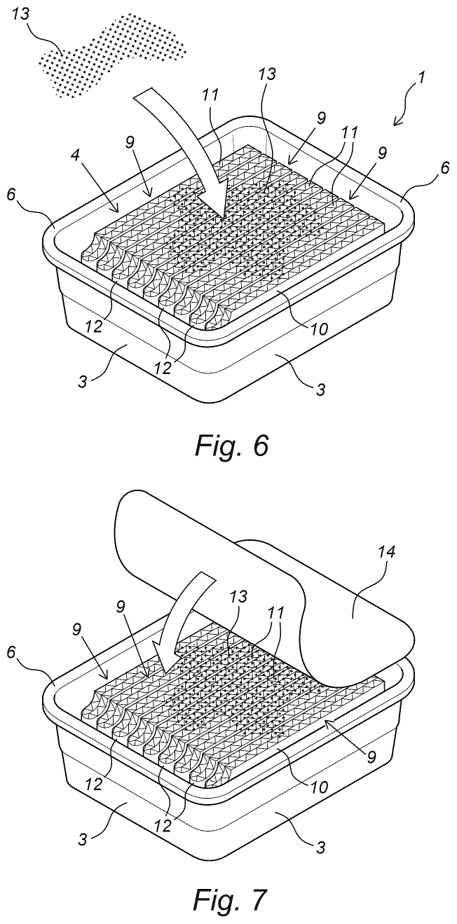

In the next step of the method, shown in FIG. 6, the beneficial arthropods are provided. In the embodiment of the invention presented in FIG. 6, the beneficial arthropods are provided on a carrier material. The carrier material together with the beneficial arthropods is indicated with reference number (13). The carrier material together with the beneficial arthropods (13) is (gently) positioned on the sides of the stacked bodies (10) of the arthropod housings (9) such that it is contact with the openings of the chambers (11). In alternative embodiments, the arthropods may be provided without a carrier material. Below experiments are presented wherein the beneficial arthropods are selected from Aphytis melinus (a parasitic wasp from the family Aphilenidae), Macrolophus Pygmaeus (family Miridae), Orius leavigatus (family Anthocoridae) and Cryptolaemus montrouzieri (family Coccinellinadae) and the selection of these beneficial arthropods and their family members is specifically within the scope of this invention. However, according to other embodiments the beneficial arthropods may also be selected outside the families of these beneficial arthropods, as already indicated above in this description of the invention and further discussed below. To temporarily reduce the motility of the arthropods, the carrier material with the beneficial arthropods (13) may be cold treated (e.g. 4-8° C.) and/or treated with CO₂ before it is positioned in the void (4) on top of the stacked bodies (10) of arthropod housings (9). This reduces escape of in particular highly motile arthropods, such as flying arthropods.

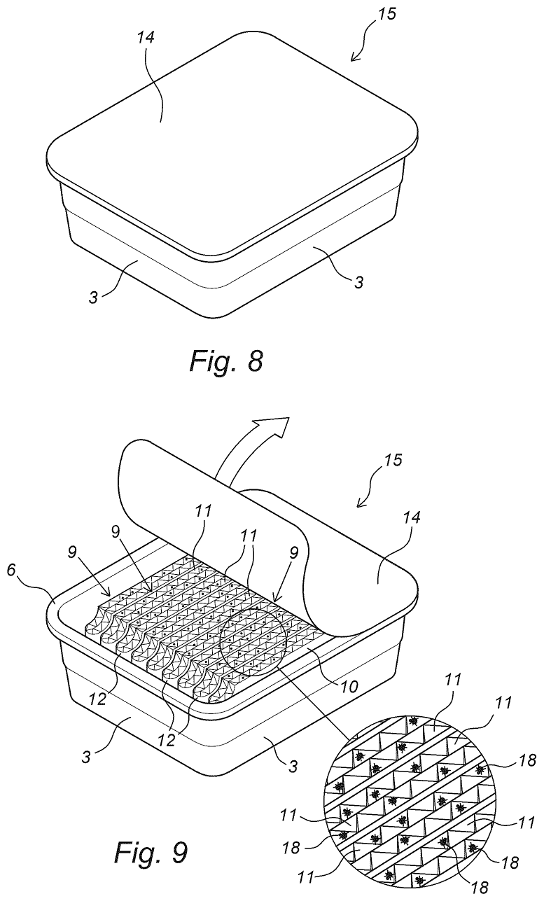

In the next step of the method, shown in FIG. 7, a closing means (14) is provided. The closing means is made from heat sealable paper (Euroflex B. V., Zwolle NL) cut to fit the dimensions of the circumference of the closure ridge (6) on the walls (3). Shortly after placing the carrier material with the beneficial arthropods (9) on the stacking of the bodies (10) of the arthropod housings (9), the void is closed, in this embodiment by heat sealing the paper (14) on the closing ridge (6), to prevent escape of the arthropods. The closed system (15), as shown in FIG. 8, can now be stored and/or transported under climatic conditions appropriate for the arthropods.

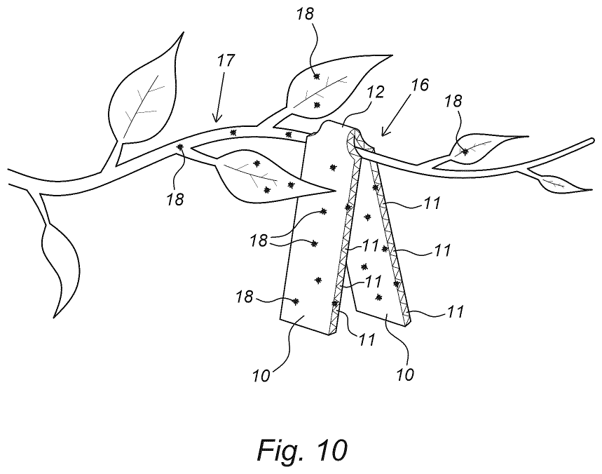

After storage at a facility and/or upon arrival in the target area after transport, the system (15) can be opened by removing the closing heat sealed paper sheet (14), as is shown in FIG. 9. If sufficient storage time has been allowed (for most arthropods around 1-4 days is sufficient), the arthropods will have distributed into the chambers (11) of different arthropod housings (9), thus forming systems for distribution of arthropods (16) according to the invention, which consist of arthropod housings (9) having beneficial arthropods in their chambers.

Distribution of the arthropods in a target area, such as a crop (16), can now be achieved by placing, such as by hanging, the systems for distribution of arthropods in the target area, as is shown in FIG. 10. By placing multiple systems for distribution of arthropods (16) on different locations in the target area, such as a crop (17), the motile individuals (18) of the arthropod population can disperse in the target area. Any arthropods remaining in the void (4) can be distributed in the target area, e.g. by gently shaking them from the void (4) or allowing them to fly away, if they have flying capability. For arthropods having flying capability, it is a mayor benefit that individuals (18) do not immediately fly away when the storage system (14) is opened, because they reside in the chambers (11). Instead, they can be transferred to a different part of the target area (17) in the releasing system (15) for release in that different part.

### EXPERIMENTS

### Experiment 1 Storage of Macrolophus pygmaeus (Rambur) (Family Miridae)

As a representative for predatory insects from the family Miridae, Macrolophus pygmaeus (Rambur) was tested for survival in the storage system of the present invention.

### Experimental Design

Survival of Macrolophus pygmaeus adults was tested in embodiments of the storage system of the invention similar to the embodiment shown in FIGS. 1-9. The embodiment used containers of mold pressed wood pulp (Earthcycle™, CKF Inc., Langley, Canada) having exterior dimensions (including the ridge) length×width×height of approximately 137×115×52 mm. The embodiment contained as the arthropod housings 9 strips of type C 4.0 mm corrugated cardboard. The spaces in the flutes of the corrugated cardboard served as chambers in the arthropod housings. The dimensions (L×W×H) of the cardboard strips were approximately 205×35×4 mm with half circular indents halfway of the strips, and cut relative to the chambers formed in the flutes, such that the direction of the chambers ran perpendicular to the longest axis of the strips (similar to what is shown in FIGS. 5-7, 9, 10). The selected number of strips had a tight fitting in the void of the containers when folded to a closed V (similar to what is shown in FIGS. 5-7, 9, 10). Five replicates were used in the test, each consisting of a storage system filled with 30 gram 1% agar, on the base of the container. The agar was covered with a fitting paperboard bottom plate resting on spacing elements positioned on the container base. The paperboard was selected to allow transmission of water vapor from the agar to the other parts of the void. After placing the 9 cardboard strips in the void, such that the chambers extended perpendicular to the base (and the openings faced to the opening of the void), for each replicate 0.75 gram product (0.50 gram Macrolophus (approx. 600 adults, 500 of normal label specification plus 20% buffer) and 0.25 gram millet husk carrier) was placed on the cardboard strips. The voids of the storage systems were closed and sealed with a covering (lid) of heat sealable paper (Koehler NexPlus® Seal Pure) using a sealing machine (MCS Sealer) at 160° C., for 8 sec.

### Procedure and Sampling

Macrolophus pygmaeus adults were collected from the mass rearing of Koppert (Berkel en Rodenrijs, NL) and mixed in the correct ratio with millet husks. The specified amounts of product containing adults of the beneficial arthropod were transferred into the storage systems using a NEWWEIGH® linear weighing machine type E104A, after anaesthetizing the arthropods with CO₂ (7 sec, 50 liter/min). After assembly and closure, the storage systems were stored at 8° C., 90% humidity and dark conditions for 4 days to simulate storage and transport. At completion of the specified storage time, each storage system was opened carefully and the carboard strips (now arthropod distribution systems, containing adult Macrolophus pygmaeus individuals in the chambers), were removed from the void of the container. From each cardboard strip the arthropods were transferred into a box by tapping them out of the chambers. The amount of dead and alive adults retrieved from the chambers of the cardboard strips were counted with standard entomological methods by trained personnel. Also, the amount of dead and alive adults on the paper closure and inside the void of the container (including under the bottom plate) were counted. This process of opening the storage systems, transferring and counting the arthropods was done inside a cold room at 8° C. to keep adults quiet.

### Data Analysis

The mean and standard error of mortality and proportion of adults on each part (strips, lid, container) were calculated from collected data.

### 3. Overall Results and Conclusions

The Macrolophus pygmaeus adults had distributed evenly into the cardboard strips (61.6±3.4 adults per strip) serving as arthropod housing. An even distribution of the arthropods over the arthropod housings is relevant as this makes it possible to distribute the arthropods more uniform in a target area. The mortality four days after packaging with the new storage system of the invention is low. The results of mortality and proportion of adults on each part is shown in table 2.

TABLE 2
Percentage (mean +/− SE) of mortality and
number of adults counted on each part of the storage
system four days after packaging and storage at 8° C.

| | | adults in | adults on | adults in |
|---|---|---|---|---|
| | Mortality | strips | lid | container |
| | (%) | (%) | (%) | (%) |
| M. pygmaeus | 6.8% ± 1.4% | 87.2% ± 1.6% | 0% ± 0% | 12.8% ± 1.6% |

In this test Macrolophus pygmaeus was tested as a representative of predatory insects form the family Miridea. This family contains several important predatory insects used for biological pest control, including but not limited to others from the genus Macrolophus, from the genus Dicyphus and from the genus Nesidiocoris, in particular Nesidiocoris tenuis (Reuter). Based on the results obtained for Macrolophus pygmaeus it may be expected that other Miridae may also be suitably stored and distributed with the system of the invention. The low mortality rate and uniform distribution over the arthropod housings seen in this first screening experiment also showed promise for tests with beneficial arthropods from other taxonomical orders.

### Experiment 2 Storage of Cryptolaemus montrouzieri (Predatory Insect from the Family Coccinellidae)

As a representative for predatory insects from the family Coccinellidae, Cryptolaemus montrouzieri was tested for survival in the storage system of the present invention.

### Experimental Design

The experiment in general was performed in analogy with experiment 1 using the same type of arthropod storage system with the same type of arthropod housings (9 in total). Five replicates were used and each replicate contained 550 adults of Cryptolaemus montrouzieri (family Coccinellidae) selected as the beneficial arthropods. Six small (0.5×0.5 cm) absorbent pads with saccharose solution (Raftisweet) were placed on top of the arthropod housings. The packages were sealed with the same type of paper as in experiment 1, using the same procedure.

### Procedure and Sampling

Cryptolaemus montrouzieri adults collected from the mass rearing of Koppert (Berkel and Rodenrijs, NL) were transferred into the storage systems after anaesthetizing the insects with CO₂ (7 sec, 50 liter/min). The storage systems were stored at 8° C., 90% humidity and dark conditions for 4 days to simulate storage and transport. After that, each storage system was opened carefully and from each cardboard strip the insects were tapped out of it into a larger box. The number of dead and alive adults was counted with standard entomological procedures by skilled personnel. Also, the amount of dead and alive adults at the lid and inside the container (at bottom plate) were counted. This process was done at ambient condition to easily recognize the dead and alive adults.

### Data Analysis

The mean and standard error of mortality and proportion of adults on each part (strips, lid, container) were calculated from collected data.

### Overall Results and Conclusions

The Cryptolaemus montrouzieri adults had distributed evenly into the carboard strips (53±1,4 adults per strip) with only little variance between the strips. The mortality four days after packaging with the new storage system is below 5%. The results of mortality and proportion of adults on each part is shown in table 3.

TABLE 3
Percentage (mean ± SE) of mortality and of number
of adults counted on each part of the storage systems
four days after packaging and storage at 8° C.

| | Mortality | Strips | Lid | Container |
|---|---|---|---|---|
| C. montrouzieri | 3.4 ± 0.4% | 87.0 ± 1.6% | 3.6 ± 1.0% | 9.4 ± 1.1% |

The new storage systems of the invention can preserve the adults of Cryptolaemus montrouzieri during storage and transport with an even distribution of adults over the arthropod housings with low mortality.

It may be expected that the results obtained for Cryptolaemus montrouzieri may be extrapolated to other predatory insects from the family Coccinellidae. This family contains several important predatory insects used for biological pest control, including but not limited to others from the genus Cryptolaemus, from the genus Adalia, in particular Adalia bipunctata, from the genus Delphastus, in particular Delphastus catalinae Horn (syn. Delphastus pusillus LeConte)), from the genus Propylea, in particular Propylea quatuordecimpunctata Linnaeus, from the genus Rhyzobius, in particular Rhyzobius lophanthae Blaisdell, from the genus, Scymnus, in particular Scymnus interruptus and from the genus Stethorus, in particular Stethorus punctillum Weise. Based on the results obtained for Cryptolaemus montrouzieri it may be expected that these and other Coccinellidae may also be suitably stored and distributed with the system of the invention.

### Experiment 3 Storage of Aphytis melinus (Parasitic Wasp, Family Aphelinidae)

As a representative for parasitic wasps, in particular from the family Aphelinidae, Aphytis melinus was tested for survival in the storage system of the present invention.

### Experimental Design

The experiment in general was performed as described for experiment 1. Four storage systems were compared in a complete randomized block design with three replicates of each system. The following storage systems were tested: 1) Standard (current) package of Koppert's Aphytis product, which is not according to the invention and 2-4 were embodiments of the storage system of the invention comparable to the systems used in experiments 1 and 2, 2) Cardboard package with food above strips (Top), 3) Cardboard package with food under strips (Bottom) and 4) food between strips (Middle). Each replicate contained 9 cardboard strips of type C 4.0 mm cardboard and was filled with ca. 12.000 adults. Standard package were sealed with a plastic lid and cardboard packages were sealed with paper adhered to the package using glue (Pritt, Henkel). In all cases, one cotton pad wetted with a 25% aqueous saccharose solution was placed inside each package to feed adults.

### Procedure and Sampling

Just collected A. melinus adults from the mass rearing of Koppert were transferred into the storage systems, and then all storage systems were maintained at 16° C., 60% RH and dark conditions for three days to simulate storage and transport to the field. After that, each storage system was opened carefully and each strip, the lid (standard) of piece of paper (carboard) and the makeup disk were transferred separately to a petri dish. Dead and alive adults inside the container were separated and transferred separately into petri dishes. This process was done inside a cold room at 8° C. to keep adults quiet, allowing to transfer each of the above-mentioned parts into the petri dishes. All petri dishes were then transferred into a freezer for 24 hours, after which the adults inside each petri dish were counted.

### Data Analysis

Treatment effects on mortality and number and proportion of adults on each part (strips, lid, container and makeup disc) were analysed using a one-way ANOVA and a Tukey test for mean separation. (α=0.05).

### Overall Results and Conclusions

Percentage of mortality three days after packaging was similar in all storage systems (Table 4), so all tested embodiments of the storage system of the invention would preserve A. melinus adults as well as the existing product. Additionally, the position of the food in the new package would not significantly affect the adult survival.

TABLE 4
Percentage (mean ± SE) of mortality and adults counted
on each part of the storage systems three days after packaging.

| | Mortality | Strips | Lid | Container | Food |
|---|---|---|---|---|---|
| Standard | 2.8 ± 0.6% a | 62.9 ± 2.8% c | 11.2 ± 1.4% a | 8.7 ± 1.1% ab | 17.2 ± 1.9% a |
| Top | 2.9 ± 0.4% a | 85.4 ± 2.1% a | 3.5 ± 0.2% b | 8.4 ± 1.5% b | 2.7 ± 0.8% b |
| Middle | 5.2 ± 2.0% a | 77.4 ± 1.9% ab | 7.0 ± 2.0% ab | 7.3 ± 2.9% b | 8.3 ± 2.9% b |
| Bottom | 3.6 ± 0.5% a | 70.7 ± 2.4% bc | 3.9 ± 0.1% b | 16.8 ± 1.2% a | 8.5 ± 1.2% b |
| Statistics | F_{3, 11} = 1.004, | F_{3, 11} = 17.001, | F_{3, 11} = 8.332, | F_{3, 11} = 5.804, | F_{3, 11} = 10.102, |
| | P = 0.440 | P < 0.001 | P = 0.008 | P = 0.021 | P = 0.004 |

A significantly lower percentage of adults was found on the paper strips in the standard product compared to those found on the carboard strips of the storage systems according to the invention with the food placed above (top) or in between (middle) of the cardboard strips. When the food source was placed on top of the cardboard strips functioning as arthropod housings, the association of adult arthropods with the strips was highest.

It may be expected that other parasitic wasps from the family Aphelinidae, and other beneficial arthropods from the order Hymenoptera can equally be stored and distributed with the systems of the invention.

### Experiment 4 Storage of Orius leavigatus (Fieber) (Family Anthocoridae)

As a representative for predatory insects from the family Anthocoridae, Orius leavigatus (Fieber) was tested for survival in the storage system of the present invention.

### Experimental Design

Survival of Orius leavigatus was tested in two alternative embodiments of the storage system of the invention similar to the embodiment used in experiments 1-3. The first embodiment contained as the arthropod housings 9 strips of type C 4.0 mm corrugated cardboard also used in experiments 1-3. The second contained as the arthropod housings 12 strips of type B 3.2 mm corrugated cardboard. The inclusion of the 3.2 mm carboard in the test was initiated after initial observations of a relatively high (but still acceptable) mortality for Orius leavigatus individuals, when using the 4.0 mm carboard as arthropod housings. Taking the relative size of the arthropods tested in the other experiments into consideration, the inventors theorized that using arthropod housings having chambers with a smaller diameter could improve the mortality rate.

For the storage systems with Type C cardboard five replicates were used and for the type B carboard three replicates. For all storage systems 30 gram of 1% agar was placed on the base of the container and the section of the void containing the agar was covered with a fitting card board bottom plate resting on spacing elements positioned on the container base. Each treatment and replicate was filled with 600 adults. The packages were sealed with heat sealable paper using a sealing machine (MCS Sealer) at 160° C., for 8 sec.

### Procedure and Sampling

Orius leavigatus adults collected from the mass rearing of Koppert (Berkel en Rodenrijs, NL) were transferred into the storage systems of the invention after anaesthetizing the insects with CO₂ (7 sec, 50 liter/min). The storage systems were stored at 8° C., 90% humidity and dark conditions for 4 days to simulate storage and transport. After storage, each storage system was opened carefully and the carboard strips (now arthropod distribution systems, containing Orius leavigatus individuals in the chambers), were removed from the void of the container. From each strip the insects were tapped out of the chambers into a box. The amount of dead and alive adults were counted. Also, the amount of dead and alive adults at the lid and inside the container (at bottom plate) were counted. The process of counting was done according to standard entomological methods and performed by trained personnel inside a cold room at 8° C. to keep adults quiet.

### Data Analysis

The mean and standard error of mortality and proportion of adults on each part (strips, lid, container) were calculated from collected data.

### Overall Results and Conclusions

It was observed that mortality four days after packaging shows lowest values for arthropod storage systems containing arthropod housings of type B (3.2 mm) cardboard (table 5). For these storage systems also more adults were associated with the chambers of the carboard strips (table 5). The adults distribute evenly throughout all cardboard strips, for cardboard Type C (4.0 mm) 44,3±1,6 adults per strip, and cardboard Type B (3.2 mm) 42,9±1,5 adults per strip. For the total package with the 9 strips Type C, it gives an average of 399 adults in total associated with the cardboard strips and for 12 strips Type B a total of 502 adults.

TABLE 5
Percentage (mean ± SE) of mortality and adults counted on different
elements of the storage systems (four days storage at 8° C.).

| | Mortality | Strips | Lid | Container |
|---|---|---|---|---|
| Type C (4.0 mm) | 17.7% ± 0.9% | 68.6% ± 2.7% | 0% | 31.4% ± 2.7% |
| cardboard | | | | |
| Type B (3.2 mm) | 8.8% ± 0.7% | 83.2% ± 1.5% | 0% | 16.8% ± 0.2% |
| cardboard | | | | |

Both embodiments of the storage system of the invention can preserve the adults of Orius leavigatus during storage and transport with a good even distribution of adults over the carboard strips. In the storage systems with type B 3.2 mm cardboard more adults were associated with the chambers of cardboard strips and mortality was lower. Adult mortality in the storage systems using Type C cardboard strip was at the borders of what is considered acceptable, while the mortality in the storage systems using Type B cardboard strips is good.

### Experiment 5 Transport Storage of Beneficial Arthropods

After the successful storage in the storage system of the invention, under passive storage conditions, of a diverse range of beneficial arthropods from families across different taxonomical orders, storage of beneficial arthropods was tested under transport conditions. For this Macrolophus pygmaeus (Rambur) (family Miridae) was used as the beneficial arthropod.

### Experimental Design and Sites

Different storage treatments were compared, each treatment was tested with five replicates over 2 repetitions. The different treatments were:
1) Control treatment: Passive storage in storage systems of the invention for 4 days at production facility of Koppert (Berkel en Rodenrijs, NL) at 8° C.;
2) Transport treatment: Transport in storage systems of the invention to Koppert facilities in Plan d'orgon (France) at 8° C. and counted after 4 days;
3) Transport treatment: Transport to Koppert facilities in Nantes (France) at 8° C. and counted after 4 days.

Each storage treatment and replicate consisted of a storage system of the type used in experiments 1-4 and filled with 30 gram 1% agar, had a cardboard bottom plate, 9 cardboard strips and filled with 0.75 product (0.50 gram Macrolophus (600 adults) and 0.25 gram millet husk). The number of adults is set at 600 to represent 500 adults as specified on products +20% buffer. To test the suitability of alternatives, packages were random sealed with 2 different types of foil paper: Paper/PE, woodfree specialty paper 72 μm, 57.0 gr/m² and Koehler NexPlus® Seal Pure using a sealing machine (MCS Sealer) at 160° C., for 8 sec. Transported storage systems were kept in an insulation box during cooled transport and temperature was monitored with a logger.

### Procedure and Sampling

Macrolophus pygmaeus adults collected from the mass rearing of Koppert. The specified amount of products consisting of Macrolophus pygmaeus adults and millet husks was transferred into storage systems using NEWWEIGH® linear weighing machine type E104A after anaesthetizing the arthropods with CO₂ (7 sec, 50 liter/min). The weighing setpoint was set on 0.75 gram. The storage systems were then stored according to the conditions specified for treatments 1-3. After completion of the storage, each storage system was opened carefully by removing the paper covering and from each cardboard strip the arthropods were tapped out of it into a box. The number of dead and alive adults was counted. Also, the number of dead and alive adults at the covering paper and inside the container (at bottom plate) were counted with standard entomological procedures by trained personnel. This process was done inside a cold room at 8° C. to keep adults quiet.

### Data Analysis

The mean and standard error of mortality were calculated from collected data.

TABLE 6

Mortality (%)

| | KBV | France |
|---|---|---|
| Macrolophus | 9.8% ± 1.5% | 5.2% ± 1.2% |

% mortality (plus SE) over two transport tests. KBV control (without transport), France (with transport to Nantes and Plan d'Orgon)

Distribution of Macrolophus pygmaeus adults across the cardboard strip arthropod housings was good in all treatments. For the treatments using “Paper/PE, woodfree specialty paper 72 μm, 57.0 gr/m²” an average of 55,9±14,7 adults was found per arthropod housing. For the treatments using KoehlerNexPlus® Seal Pure a comparable average of 60,0±13,5 adults was found per arthropod housing.

### Experiment 6 Distribution of Beneficial Arthropods

Population build-up in a crop by Macrolophus pygmaeus adults released with the new storage systems of the invention was tested and compared with that of Macrolophus pygmaeus adults released from the packaging of two prior art products. The first prior art product was the existing Mirical® product of Koppert (Berkel en Rodenrijs, NL), the second the Macrolophus System of Biobest (Westerlo, BE). The prior art products, according to the specification, contain 500 adults. The storage systems of the invention containing Macrolophus pygmaeus were produced as described in example 1 with the same specified number of adults.

### Trial Design

The experiment was set out to be conducted in weeks 19-27 (2022) in a commercials tomato crop grown in a greenhouse located in Rotterdam (The Netherlands). The experiment is based on how Macrolophus pygmaeus in general is released in practice in greenhouse tomato cultivation.

Briefly described the main points of this general methodology are:
Per trellis, 1 release row is selected with several release sites at an equal distance. Approx. 1 release site every 10 metres.
The release is done 1 to 2 times per week. On average between 1 to 2 Macrolophus/m².
The row is fed weekly with approx. 1 box Entofood 10 g/100 m row until the first new adults (6 to 8 weeks after release).
Depending on the time of year, fewer leaves are cut away at the base of the plant during the first generation to avoid too many young Macrolophus being discarded with the old leaves.

The grower and consultant evaluate the product in the field by numbers of individuals they observe on the plants. During the population build-up phase, about 50-100 leaves are scored in a release row to see how many Macrolophus individuals are present. Later, this is also occasionally done in other areas of the greenhouse. This is a rough method to assess how the population of Macrolophus is doing in the plant/greenhouse.

In this trial design, a release of 1× ca.1 Macrolophus/m² was chosen. Given the time of year (week 19-27), the reduced leaf-cutting was not done. This was not necessary, because of the calm growth of the plants and the rapid development of Macrolophus this time of year. Furthermore, about 2×Entofood 10 g per row was fed weekly. The amount of Entofood is more than what a grower will normally use, but this was done to avoid that food could be a limiting factor for Macrolophus development.

The three treatments were randomly divided over 6 treatment slots of 2 adjacent rows of plants, each row being about 100 metres long and each treatment slot being separated from the other treatment slots by 3 buffer rows of plants. The actual layout of the rows in the experiment was as follows:
BBT3T3BBBT2T2BBBT1T1BBBT3T3BBBT1T1BBBT2T2BB

Wherein B is a buffer row of completely untreated plants, T1, T2 and T3 are rows wherein at a release point Macrolophus individuals are released from respectively the storage system according to the invention (T1), the Mirical® (T2) product and the Macrolophus system from Biobest (T3). Individual rows were separated by an aisle.

Over the length of each treatment slot 10 evenly spaced release patches with 2 release plants were designated, such that these release patches were also equally divided over the two treatment rows (5 release patches per treatment row). The release patches had the following layout SPPRPPSPPRPPS, wherein S is a sampling plant, P is a buffer plant and R is a release plant. Each treatment thus contained 20 release patches, 40 release points and 60 sampling points. For each treatment slot the Macrolophus individuals from 2 packages of the tested storage/release system were evenly distributed over the 20 release points, thus a total of 4 packages was used per treatment.

### Release

### New Storage System of Invention

For this treatment 1 cardboard release system was hung over the petiole of the release plant on the 1st mature leaf. After placing all 9 cardboard release systems in a release plant, he contained holding them was placed in a release plant in the same place as the cardboard release systems, such that any adults remaining therein could disperse into the crop. Approximately 50 Macrolophus were introduced per release plant.

### Mirical®

The material from the scatter bottles was evenly distributed in Diboxes (Koppert, Berkel en Rodenrijs, NL) hung over the petiole of the release plant on the 1st mature leaf. Approximately 50 Macrolophus were introduced per release plant.

### Biobest Packaging

Equal distribution from this product was a lot trickier, as the product consists of loose adult Macrolophus individuals plus some paper strips in a container. The introduction was done by carefully opening the lid and releasing ca. 50 Macrolophus individuals at the release plant in the same place as the other treatments. Further, on some plants paper strips with ca. 50 Macrolophus were placed at the same position in the plant as in the other treatments.

### Product Assessment

A visual assessment was done during product release. None of the products had remarkably high numbers of dead individuals.

### Supplementary Feeding

Before releasing the Macrolophus individuals, the release rows were treated with Entofood (Koppert, Berkel en Rodenrijs, NL). This was done to prevent the Macrolophus from spreading over a large area en masse. Weekly during the trial, the treated rows were fed Entofood 10 gr. The Entofood was blown from the aisle over the head with a Mini airbug over the rows both left and right. Approximately 2 pots of Entofood per aisle (1 per row).

### Counting Methodology

Weekly counts were made on 3 marked sampling plants (S) per release patch. For each sampling plant, 9 mature leaves were scored from above for adults, young nymphs (L1-L2) and old nymphs (L/3-L5). During the counts, further distinctions were also made into Upper (leaves 1-3), Middle (leaves 4-6) and Lower (leaves 7-9).

### Climate Conditions During the Trial

The trial was conducted under relatively favourable conditions. Weather conditions were constant with many sunny days. Daytime temperatures were warm with peaks of >30° C. in the greenhouse. At night, it was then relatively cold (sometimes <10° C.) because hardly any heating was done due to high gas prices. The average temperature was about 21.5° C.

### Results

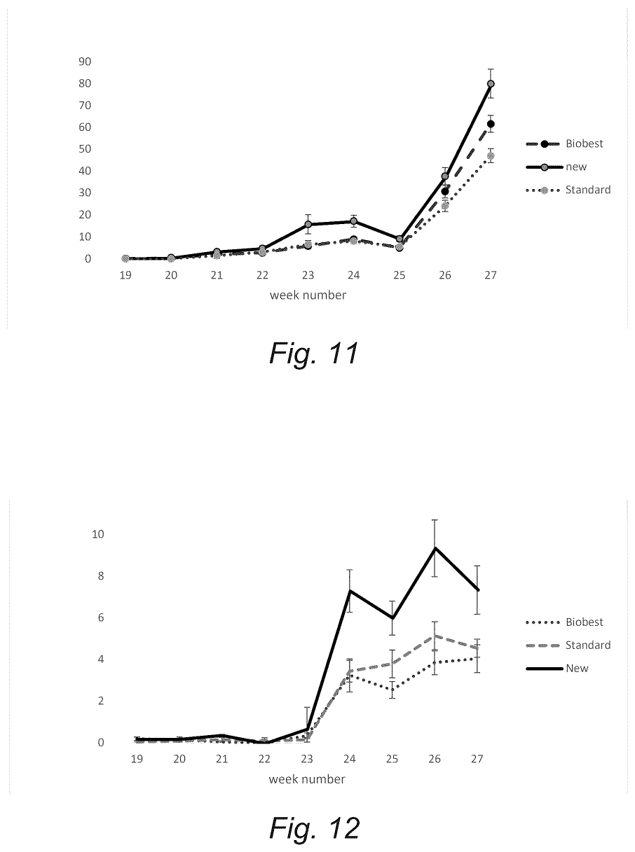

The development of the number of counted Macrolophus individuals after release from the storage system of the invention (new), from the Mirical® product (standard) and from the Biobest product is shown table 7 below. The table shows numbers separately for L1-L2, L3-L5 instars and adults. FIG. 11 graphically presents the development in time of the average number of Macrolophus individuals of all life stages counted per replicate (20 per treatment), whereas FIG. 12 shows a graphic presentation for the average number per replicate of adults counted over time.

TABLE 7

| | | Biobest | Mirical ® | Invention |
|---|---|---|---|---|
| week 20 | L1-L2 | 7 | 0 | 8 |
| | L3-L5 | 0 | 0 | 0 |
| | Adult | 3 | 2 | 4 |
| week 21 | L1-L2 | 41 | 28 | 61 |
| | L3-L5 | 0 | 0 | 0 |
| | Adult | 1 | 3 | 8 |
| week 22 | L1-L2 | 29 | 42 | 32 |
| | L3-L5 | 27 | 19 | 67 |
| | Adult | 0 | 2 | 0 |
| week 23 | L1-L2 | 12 | 20 | 92 |
| | L3-L5 | 99 | 110 | 212 |
| | Adult | 7 | 3 | 14 |
| week 24 | L1-L2 | 14 | 8 | 20 |
| | L3-L5 | 102 | 86 | 180 |
| | Adult | 65 | 69 | 146 |
| week 25 | L1-L2 | 3 | 3 | 11 |
| | L3-L5 | 48 | 33 | 54 |
| | Adult | 51 | 76 | 120 |
| week 26 | L1-L2 | 515 | 372 | 548 |
| | L3-L5 | 25 | 10 | 22 |
| | Adult | 77 | 103 | 187 |
| week 27 | L1-L2 | 1056 | 792 | 1414 |
| | L3-L5 | 99 | 62 | 43 |
| | Adult | 81 | 91 | 147 |

The data shows that development of the Macrolohus population released from the storage systems of the invention meets the requirements and is clearly improved over that of the other two tested storage systems. There are major differences in the development of the first generation after release. In the first generation, the system of the invention gives >13000 more offspring than the Standard packaging and the Biobest packaging (see week 23-24). Releasing the Macrolophus individuals using the cardboard strip releasing systems from the invention is also very easy and fast. The cardboard strip is also a handy marker for the grower of a release site.

It may be expected that similar benefits will be obtained with similar predatory insects from the family Miridae, including but not limited to others from the genus Macrolophus, from the genus Dicyphus and from the genus Nesidiocoris, in particular Nesidiocoris tenuis (Reuter).

In view of the experimental proof of successful storage (and distribution) of beneficial arthropods selected from a wide range of taxonomic orders, it may be expected that similar effects may be expected with beneficial arthropods from other families from the same orders and even with beneficial arthropods from other orders.

1. System for releasing beneficial arthropods comprising:
an arthropod housing comprising a body, preferably a plate formed body, provided with a number of chambers enclosing an interior space, wherein the shape of the interior space of the number of chambers preferably is such that the dimensions in at least one direction are smaller than the dimensions in another direction, wherein each chamber further has a number of openings suitable to provide access of the arthropods to the interior space, and the chambers comprise individuals, preferably motile individuals, most preferably adult motile individuals, of a beneficial arthropod species.

2. System according to claim 1, wherein the body comprises a plurality of chambers.

3. System according to claim 1, wherein the interior space of the number of chambers has an elongated shape, and wherein more preferably the body is provided with a plurality of parallelly positioned chambers having an opening on at least one end of the number of elongated chambers.

4. System according to claim 1, wherein the body of the arthropod housing has a plate form, and wherein preferably the longest axis of the chambers is essentially perpendicular to the normal vector of the plane of the body.

5. System according to claim 1, wherein the average length of the bodies of individuals of the selected beneficial arthropod species is L, the diameter of the elongated chambers of the arthropod housing is D and D/L is >1.1, such as >1.2, >1.5, preferably >2.5.

6. System according to claim 1, wherein the average width of the bodies of individuals of the selected beneficial arthropod species is W and the diameter of the elongated chambers of the arthropod housing is D and D/W is >1.2, more preferably >2.5.

7. System according to claim 1, wherein the beneficial arthropods species is a biological control agent, preferably selected from Coccinellidae, Staphylinidae, Cecidomyiidae, Syrphidae, Anthocoridae, Lygaidae, Miridae, Nabidae, Pentatomidae, Ampulicidae, Aphelenidae, Bethylidae, Braconidae, Diapriidae, Encyrtidae, Eulophidae, Figitidae, Mymaridae, Platygastridae, Pteromalidae, Scelionidae, Trichogrammatidae, Chrysopidae, Aeolothripidae, Phlaeothripidae, Thripidae, Phytoseiidae, Ascidae, Laelapidae, Macrochelidae, Parasitidae, Tydeidae, Cheyletidae, Cunaxidae, Erythraeidae, Stigmaeidae, Anystidae and more preferably selected from Coccinellidae, Anthocoridae, Miridae, Aphelenidae.

8. System according to claim 1, wherein the beneficial arthropods species is selected from:
the family Anthocoridae, such as from the genus Anthocoris, e.g. Anthocoris nemoralis (Fabricius), Anthocoris nemorum (Linnaeus), from the genus Orius, e.g. Orius albidipennis (Reuter), Orius insidiosus (Say), Orius laevigatus (Fieber), Orius majusculus, Orius sauteri (Poppius), Orius strigicollis (Poppius) or Orius tristicolor (White);
the family Miridae, such from the genus Dicyphus, e.g. Dicyphus errans (Wolff) or Dicyphus hesperus Knight, from the genus Macrolophus, e.g. Macrolophus pygmaeus (Rambur), from the genus Nesidiocoris, e.g. Nesidiocoris tenuis (Reuter).

9. System for storing beneficial arthropods, said system comprising:
I. a container having a base and a number of walls extending from the base, said number of walls surrounding a void;
II. a number of arthropod release systems according to claim 1 positioned in the void;
III. preferably a food source for the individuals of the beneficial arthropod species;
IV. preferably a water source suitable for the individuals of the beneficial arthropod population, optionally together with a separation means, suitable to separate the water source in a separated part of the void;
V. closing means closing the void, preferably closing means cooperating with a number of the walls.

10. System according to claim 9, wherein a plurality of arthropod release systems is positioned in the void, such that openings of the chambers, preferably openings of chambers of arthropod housings of all the release systems, are facing the opening of the void and wherein preferably the plurality of arthropod release systems has a tight fitting in the void.

11. System according to claim 9, wherein the arthropod release systems have a body having a plate form, and the arthropod release systems are positioned in the void in a stacking.

12. System according to claim 9, wherein the three-dimensional configuration of the interior space of a number of chambers has an elongated shape, the body is provided with a plurality of parallelly positioned chambers having an opening on at least one end of the elongated chamber and wherein preferably the body has a plate form and the longest axis of the chambers is essentially perpendicular to the normal vector of the plane of the body.

13. Method for producing a system for storing and/or releasing beneficial arthropods, said method comprising:
A. providing a container having a base and a number of walls extending from the base, said walls surrounding a void;
B. providing a number of arthropod housing devices as defined in claim 1;
C. providing individuals, preferably motile individuals, of a population of the beneficial arthropod, optionally on a carrier material;
D. preferably providing a food source for the individuals of the beneficial arthropod species and placing the food source in the void;
E. preferably providing a water source suitable for the individuals of the beneficial arthropod population optionally together with a separation means, suitable to separate the water source in a separated part of the void and placing the water source in the void;
F. placing the number of arthropod housing devices in the void;
G. placing the individuals of the beneficial arthropod population, optionally on a carrier material, in the void;
H. providing closing means suitable to close the void and closing the void with said closing means.

14. Method for biological pest control, preferably in crop protection, comprising providing a number of systems according to claim 1 to a target area, such as a crop, preferably by hanging the number of systems in the target area.

15. Use of the system according to claim 1 in pest control, preferably in crop protection.
